(19) United States
(12) Patent Application Publication
(10) Pub. No.: US 2026/0363676 A1
(43) Pub. Date: Nov. 19, 2026

(54) TREATMENT METHODS BY USING A CATHETER FOR ISOLATING AN INTERIOR OF A MAMMAL HOLLOW ORGAN (VARIANTS)

(71) Applicant: PANDICA LTD, London (GB)

(72) Inventors: Aleksei Arievich KASHINTSEV, Saint Petersburg (RU); Vitali Yurievich PROUTSKI, Saint Petersburg (RU); Sergey Vladimirovich ANISMOV, Saint Petersburg (RU); Oleg Konstantinovich GRANSTREM, Saint Petersburg (RU)

(73) Assignee: PANDICA LTD, London (GB)

(21) Appl. No.: 19/103,169

(22) PCT Filed: Dec. 31, 2022
(86) PCT No.: PCT/IB2022/062941

§ 371 Date: Feb. 11, 2025

(30) Foreign Application Priority Data
Aug. 12, 2022 (RU) 2022122019
Oct. 6, 2022 (RU) 2022126129
Dec. 15, 2022 (RU) 2022132961

(51) Int. Cl. A61B 10/00; A61M 25/10

(52) U.S. Cl. CPC A61B 10/0045; A61M 25/1011; A61B 2010/0061; A61M 2025/1052

(57) ABSTRACT

Uses for treatment of bleeding into an esophageal or intestinal lumen, for treatment of an esophageal wall fistula or perforation, for treatment of an intestinal wall fistula or perforation, for treatment, severity assessment, prognosis and monitoring of acute pancreatitis and for treatment of small intestine wall lesion are disclosed herein related to a catheter for isolating a region in a hollow organ of a mammal. The used catheter at least comprises an elongate body, two inflatable balloons disposed along the body, a functional channel extending in the body and comprising a functional opening provided in the body between the balloons, and a second channel extending in the body, wherein the second channel is provided at opposite ends with an inlet and an outlet provided both in the body outside the isolated interior defined by the balloons.

### FIELD OF THE INVENTION

The present invention relates to medicine, and more particularly to medical devices used to diagnose, monitor and/or treat inflammatory, autoimmune, infectious, benign and/or malignant diseases, that occur in hollow organs and other mammal organs that are connected to the hollow organs, in particular diseases of pancreas, bile ducts, liver, gastrointestinal tract, and also defects and injuries of mamal hollow organs, fistulas, strictures, aneurismal and diverticular dilatations, and other diseases of the mammal hollow organs.

### BACKGROUND

Known in the art are various devices and appliances used to diagnose and/or treat inflammatory, autoimmune, infectious, benign and/or malignant diseases that occur in hollow organs of a mammal, in particular diseases of pancreas, bile duct diseases, liver diseases, and also gastrointestinal wall defects, injuries of the wall of a hollow organ, aneurismal and diverticular dilatation, strictures of hollow organs, bleeding of hollow organs and some other diseases of hollow organs, and diseases of organs connected to hollow organs.

According to guidelines for diagnosing and treating a pancreatic cancer, main techniques allowing for a histological type of neoplasm to be confirmed are fine-needle percutaneous core-biopsy, fine-needle functional biopsy, cytological analysis of an epithelial scraping (also referred to in the art as a brush-biopsy), diagnostic laparoscopy with a biopsy, and cytological analysis of washings obtained from an abdominal cavity during laparoscopy or laparotomy (Pancreatic adenocarcinoma guidelines. National Cancer Comprehensive Network. 2019 Version 2.2019). These officially recommended biopsy techniques are based on different principles, and therefore they cannot be regarded as the closest prior art for the present invention.

In particular, known in the art is an ultrasonic method for studying pancreas (Minko A.B. Complex beam diagnostics of pancreas diseases/A.B. Minko, B.C. Pruchansky, L.I. Korytova.—SPb: Hippokrat, 2001.—134 p.; Martínez-Noguera A., Montserrat E., Torrubia S., et al., 2001. Ultrasound of the pancreas: update and controversies. Eur Radiol 11:1594-1606). The ultrasonic method is based on the assessment of changes in the reflection and absorption of waves from an organ tissue and allows for gland contours, liver contours and alternations of hyperechoic or hypoechoic zones to be determined. Furthermore, the ultrasonic method allows for determination a Wirsung duct formation and its diameter, a bile duct formation and its diameter, and other formations and their diameters. The main disadvantage of an ultrasonic method is in that examination result depends on a specialist's qualification and an apparatus' resolution. In this case, the sensitivity of the ultrasound examination is 70-80%. If a size of the formation is less than 1.5 cm, the ultrasonic method has strongly reduced effectiveness. In particular, the ultrasonic method does not allow for intraductal neoplasms to be detected, the pancreatic duct to be completely evaluated, a biochemical composition of pancreatic juice and bile to be evaluated, and pancreatic juice and bile to be cytologically examined.

Furthermore, known in the art is a method of endoscopic ultrasound examination based on the same principle as the above-described ultrasonic method (see Ogawa M., Kawaguchi Y., 2011, Iglesias-Garcia J., 2012). An advantage of the endoscopic ultrasound examination over the ultrasonic method is in that biopsy may be performed in close proximity to a site of interest. A convex probe, if any, allows for a suspicious formation to be examined with a fine-needle functional biopsy followed by a cytological examination. Disadvantages of the endoscopic ultrasound examination are the following: necessity to have a highly skilled specialist, necessity to perform anesthetic aid, high costs, and lack of possibility to perform examination in some cases due to some anatomical features. Furthermore, endoscopic ultrasound examination is not suitable for performing cytologic diagnostics of liver and of bile ducts and allows only for a small amount of cytological material to be obtained by performing aspiration, so that it is difficult to interpret the obtained cytological material, causing a high proportion of false-positive results and false-negative results. Furthermore, the endoscopic ultrasound examination does not allow for a qualitative analysis of the composition of bile or pancreatic juice.

Furthermore, known in the art is a fine-needle biopsy, wherein the fine-needle core-biopsy is always used in combination with the above-described ultrasonic method and the above-described endoscopic ultrasound examination and allows for a material for histological examination to be obtained (Hruban R.H., Takaori K., Klimstra D.S. An illustrated consensus on the classification of pancreatic intraepithelial neoplasia and intraductal papillary mucinous neoplasms. Am J. Surg. Pathol. 2004. V.28 (8) P.977-87). The fine-needle biopsy is a main method used to histologically verify pancreatic diseases. Disadvantages of the fine-needle biopsy are possible complications: bleeding, formation of fistulas, abscesses, dissemination of cancer cells along a puncture channel, and also uninformative, false-positive or false-negative results of histocytological examinations. Furthermore, the fine-needle biopsy is not suitable for diagnosing diseases of the bile ducts and gallbladder and does not allow for the laboratory evaluation of bile or pancreatic juice.

Furthermore, known in the art is a method of spiral computer-assisted tomography with an intravenous contrast (MSCT) (Callery M.R. et al., 2009; Klaub M. et al., 2009). The spiral computer-assisted tomography is based on the computer-assisted processing of thin “slice” tomograms, assessment of the degree of absorption of a contrast agent by the tissue of a gland or tumor, and the measuring of a diameter of the ducts. The spiral computer-assisted tomography allows for the visualization of formations and making a decision on whether an acute or chronic inflammatory change of corresponding organ is observed based on changes in organ densitometric parameters. Disadvantages of the spiral computer-assisted tomography are significant decrease in its sensitivity when the formation's size is less than 1.5 cm, and low sensitivity and specificity when diagnosing intraductal neoplasms. Furthermore, spiral computer-assisted tomography does not allow for a disease to be histologically confirmed and does not allow for pancreatic secretion to be analized.

Furthermore, known in the art is an endoscopic retrograde cholangiopancreatography (ERCP) method (Kawaguchi Y., Ogawa M., Omata F. Randomized controlled trial of pancreatic stenting to prevent pancreatitis after endoscopic retrograde cholangiopancreatography. World Journal of Gastroenterology 2012. Vol. 18 (14). P. 1635-1641). In the endoscopic retrograde cholangiopancreatography, focal formations and calculi are revealed in the form of filling defects. Disadvantages of the endoscopic retrograde cholangiopancreatography are the need for anesthetic aid, a number of limitations preventing certain manipulations, and injuries associated with the procedure, potentially causing destructive pancreatitis or acute cholangitis.

Furthermore, known in the art is an endoscopic aspiration method for aspirating pancreatic juice from duodenum interior by stimulating excretory function of pancreas with Chirhostim™, which is a synthetical analogue of secretin (Suenaga M., Sadakari Y., Almario J.A., et al. Using an endoscopic distal cap to collect pancreatic fluid from the ampulla. Gastrointest Endosc. 2017; 86 (6): 1152-1156; Kanda M., Sadakari Y., Borges M., et al. Mutant TP53 in Duodenal samples of pancreatic juice from patients with pancreatic cancer or high-grade dysplasia. Clin Gastroenterol Hepatol. 2013; 11 (6): 719-730). The endoscopic aspiration method is used for sampling a material followed by cytological examination and/or molecular genetic analysis thereof. Disadvantages of the endoscopic aspiration method are as follows: lack of public acceptance, and limitation of the amount of sampled pancreatic secretion by duration of drug's action by short duration of the procedure. It is of note that composition of pancreatic juice may change when secretin or its analogues is administered, so that the sampled pancreatic secretion in this case does not have its natural composition, and therefore it is not impossible to evaluate the real composition and secretion clearance of pancreatic juice since the function is stimulated by the drug. The endoscopic aspiration method is also not suitable for collecting and analyzing bile.

Furthermore, known in the art is a magnetic resonance imaging (MRI) method used for determining neoplasms and inflammatory changes of pancreas and/or bile ducts (Akisik MF, Sandrasegaran K., Aisen AA Dynamic secretin enhanced MR cholangiopancreatography. RadioGraphics. 2006. Vol.26. P.665-677). The magnetic resonance imaging is a non-invasive method and provides an increased sensitivity and specificity when used in combination with radiographic opacification or stimulation of excretory function of pancreas. The disadvantage of the magnetic resonance imaging is the impossibility of evaluating a composition and clearance of bile or pancreatic juice and performing cytological examination thereof.

Furthermore, known in the art is a method of performing a nasopancreatic or nasobiliary drainage followed by the isolated sampling of a substrate (Handa K., Minami T., Shimizu A., et al. Roles of ERCP in the Early Diagnosis of Pancreatic Cancer. Diagnostics 2019, 9 (1), 30; Budzinsky S.A., Shapovalyants S.G., Fedorov E.D., Shabrin A.V. Endoscopic transpapillary pancreatic stenting in the treatment of pancreatic fistulas (with a commentary by A.G. Krieger). Journal named after N.I. Pirogov. 2017; (2): 32-44). The nasopancreatic or nasobiliary drainage is used both for treating inflammatory pancreatic diseases and for performing their cytological verification, wherein the nasopancreatic or nasobiliary drainage is suitable for compositional analysis of biofluids. Furthermore, the nasopancreatic or nasobiliary drainage is a single method allowing for pancreatic juice and bile to be independently sampled. A disadvantage of the nasopancreatic or nasobiliary drainage is the complexity of implementation. For the first time, the use of the nasopancreatic or nasobiliary drainage was described in 1980, however the nasopancreatic or nasobiliary drainage is not a routine method and used only in highly specialized institutions, in particular for solving a limited range of tasks, primarily for treating acute pancreatitis. In some cases, particularly due to the presence of some anatomical features, the nasopancreatic or nasobiliary drainage may not be implemented and may cause various complications such as pancreatitis, cholangitis and bleeding.

Furthermore, known in the art is a two-channel Dreiling tube used for performing duodenal intubation (Stevens T., Conwell DL, Zuccaro G., et al. A prospective crossover study comparing secretin-stimulated endoscopic and Dreiling tube pancreatic function testing in patients evaluated for chronic pancreatitis. Gastrointestinal Endoscopy. 2008. 67 (3). P. 458-466; Pollack BJ, Grendell JH. Where have all the dreiling tubes gone? Am J Gastroenterol. 2006 February; 101 (2): 356-359). The Dreiling tube allows for a non-invasive sampling of pancreatic juice and bile followed by a biochemical test and/or cytological examination thereof and by determination of daily clearance. A disadvantage of the Dreiling tube is that it does not allow for the selective sampling of the content of duodenum, leading to the retrograde sampling of intenstinal contents. The passive nature of sampling of excreta leads to its partial loss in distal sections due to the peristalsis of duodenum. However, it is of note that passive positioning of the tube, achieved by the weight of the olive in a distal part of the tube and peristalsis of gastrointestinal tract, leads to excessive time required for positioning of the tube and the need to perform radiographic correction of its position, wherein it is difficult to provide an appropriate positioning of the tube's channels for sampling pancreatic juice and bile. The use of stimulating drugs, such as, for example, secretin, cannot be regarded as an adequate solution since they only increase the bicarbonate buffer excretion provided by cells of the pancreas, i.e. only the activity of the epithelial cells covering the ducts is stimulated, and most of the exocrine gland apparatus remains inactive. Therefore, the Dreiling probe does not allow for stimulation of the function of adenocarcinomas, mucinous neoplasms and neuroendocrine tumors. Furthermore, the Dreiling tube is an alternative to the endoscopic retrograde cholangiopancreatography and may be used only to diagnose chronic pancreatitis, wherein intraductal mucinous and cystous formations and carcinomas cannot be revealed in situ by using the Dreiling tube. Furthermore, when the Dreiling tube is used, it is quite difficult to evaluate the choleresis due to the passage of a part of bile or pancreatic juice through the duodenum beside the Dreiling tube.

Furthermore, known in the art is a nasopancreatic stent inserted into the main duct of pancreas by using an endoscopic technique (Osnes M., Petersen H., Schrumpf E. Comparison of juice obtained during duodenal aspiration and cannulation of the main pancreatic duct after stimulation with exogenous secretin in man. Scand J Gastroenterol. 1978; 13 (4): 453-8; Minami T., Hanada K., Hirano N., et al. Clinical Usefulness of Serial Pancreatic-Juice Aspiration Cytological Examination and Endoscopic Ultrasound-Guided Fine-Needle Aspiration in Small Pancreatic Cancer. 152, Issue 5, Supplement 1, Page S897; Bi Y., Ji B., Raimondo M. How to suction pancreatic juice from the duodenum: Endoscope, catheter, or cap-assisted No. 86 (6). 2017 Gastrointestinal endoscopy. P. 1157-1159). During an endoscopic papilosphincterotomy, the nasopancreatic stent allows a probe to be inserted and, therefore, allows for pancreatic secretion to be obtained. The disadvantage of the nasopancreatic stent is its traumatic nature, leading to the development of pancreatitis, cholangitis or obstructive jaundice. In 3-10% of cases, an endoscopic retrograde cholangiopancreatography may cause acute pancreatitis, so that in this case all patients have to go through a special preventive therapy. Furthermore, during the papilosphincterotomy, a large vessel may be damaged, thereby causing bleeding. It is to note that the nasopancreatic stent may be used only in highly specialized centers by a specialist experienced with the procedure.

Furthermore, known in the art is a dual-lumen duodenal probe inserted through a nasal passage under control of an endoscope, allowing for the duodenal probe to be advanced through a pyloroduodenal area (Bi Y., Ji B., Raimondo M. How to suction pancreatic juice from the duodenum: Endoscope, catheter, or cap-assisted? 86 (6). 2017 Gastrointestinal endoscopy. P. 1157-1159; Go VL, Hofmann AF, Summerskill WH. Simultaneous measurements of total pancreatic, biliary, and gastric outputs in man using a perfusion technique. Gastroenterology 1970; 58: 321-328). A disadvantage of the duodenal probe is the impossibility of selective sampling of pancreatic juice and bile, and lack of a barrier for mixing of the excreta with intenstial and gastric contents, which leads to activation of pancreatic enzymes and digestion of biological material in the probe. Furthermore, it is of note that design of the duodenal probe does not allow one to influence the outflow of pancreatic juice from the pancreas, so that in order to obtain the juice and perform its examination, pancreas has to be stimulated by drugs such as secretin.

Furthermore, known in the art is a pancreatic juice aspiration device configured to aspirate pancreatic juice by using an endoscope formed as a cap fitted on a fibroscope, wherein the cap allows for the pancreatic secretion to be collected after stimulation of the organ's exocrine function (Suenaga M, Sadakari Y, Almario JA, et al. Using an endoscopic distal cap to collect pancreatic fluid from the ampulla (with video. Gastrointest Endosc 2017; 86: 1152-1156). Disadvantages of the aspiration device are as follows: the complexity of the procedure since it can be implemented only in highly specialized centers, and a small amount of the collected pancreatic secretion (wherein the available amount of pancreatic secretion is a critical aspect of a molecular test). In this case, the procedure based on the use of the aspiration device is performed by using anesthetic aid, wherein the procedure cannot be performed for a long time.

Known in the art is a method of diagnosing viral hepatitis in blood by using serological and molecular genetic methods. Nevertheless, in 20% of cases, a disease remains unverified since it is conditioned by a life cycle of a virus and by its tropism both to hepatocytes and to the epithelium of the bile ducts, causing viral cholangitis and the disease chronization (Shakhgildyan I.V., Mikhailov M.I., Onishchenko G.G. Parenteral viral hepatitis (epidemiology, diagnosis, prevention). Moscow: GOU VUNMTS MZ RF, 2003; Burgart LJ Cholangitis in Viral Disease. Mayo Clinic Proceedings; 1998. V. 73(5 ); 479-482). The diagnostic method does not allow for obtaining a bile for further laboratory analysis.

Known in the art is a method of diagnosing diseases of the liver (viral hepatitis, autoimmune hepatitis, sclerosing cholangitis, liver tumors) by performing a percutaneous biopsy followed by histological and molecular genetic analysis (Bunt E.M. Liver Biopsy Diagnosis of Hepatitis: Clues to Clinically-Meaningful Reporting. Mo Med. 2010; 107 (2): 113-118). In some cases, such a method leads to complications (bleeding, biliary peritonitis). At the same time it does not allow one to reveal pathologic changes in about 30% of cases. Furthermore, such a method is used exclusively for primary diagnostics, and practically never used repeatedly, for example for monitoring the dynamics or development and progression of a disease.

Furthermore, known in the art is a method of treating acute pancreatitis by stenting the main pancreatic duct to restore the efflux of enzymes of the pancreas. Such a method is used for treating and preventing pancreatitis after performing the endoscopic retrograde cholangiopancreatography (Mozharovsky V.V., Mutnykh A.G., Zhukov I.N., Mozharovsky K.V. Stenting of the main pancreatic duct influences the treatment results obtained for patients with an acute pancreatitis. Surgery. Journal named after N.I. Pirogov. 2019; (9): 13-17; Dumonceau JM., Andriulli A., Elmunzer BJ., et al. Prophylaxis of post-ERCP pancreatitis: European Society of Gastrointestinal Endoscopy (ESGE) Guideline—updated June 2014. Endoscopy. 2014 September; 46 (9): 799-815). A disadvantage of the method is in that stenting procedure can be performed only in highly specialized centers, wherein in some cases the procedure cannot be performed at all due to anatomical features of patient's duct system. Furthermore, the procedure does not solve the problem of pancreatitis due to the disruption of the outflow of pancreatic juice associated with paresis of duodenum.

Furthermore, intestinal fistulas occur on average in 1-3% of patients after performing operations on abdominal cavity organs (Smotrin I.S. Obturating agents for treating gastrointestinal fistulas. Journal of the State Medical University for Practicing Physicians.—2007.-No. 4—P.160-163). The overall mortality associated with this pathology ranges from 16.5% to 57.5%, and the postoperative mortality associated with this pathology ranges between 10% and 21.4%, wherein the greatest mortality is observed for disembodied intestinal fistulas and ranges between 36% and 71.7%. The mortality barely reaches 4% for embodied intestinal fistulas.

Known in the art is a method of treating gastrointestinal fistulas by using systems for producing a negative pressure, wherein the method is based on the constant evacuation of all pathologic discharge from abdominal cavity, thereby healing defects (Bobkiewicz A., Walczak D., Smoliński S. et al. Management of enteroatmospheric fistula with negative pressure wound therapy in open abdomen treatment: a multicenter observational study. Int Wound J. 2017 February; 14 (1): 255-264; D'Hondt M., Devriendt D., Van Rooy F. et al. Treatment of small-bowel fistula in the open abdomen with topical negative-pressure therapy. Am J Surg. 2011; 202 (2): e20-24). Furthermore, in case when the method is used, statistics collected by the same authors indicate that such defects are not closed (not healed) in 30-47% of cases due to contents constantly produced and received from a lumen of a hollow organ.

Furthermore, there are a large number of different obturator-like devices aimed at disconnecting the lumen of a hollow organ with a defect in a wall. However great variety of such devices suggests the difficulty of their implementation and achieving desired effects. Furthermore, all such well-known devices are aimed only at disconnecting the lumen of a hollow organ with a defect in a wall and do not aim to impact an area adjacent to the wall defect (Vitsyn B.A., Blagitko E.M. Formed and unformed external intestinal fistulas.—Novosibirsk: Nauka.—1983.—142 pp.; Makarenko T.P., Bogdanov A.V. Gastrointestinal fistulas.—M.: Medicine.—1986.—144 pp. USSR AS 764685, M. class A 61 M 27/00. Obturator for temporary closure of a gastrointestinal fistula/V.M. Udod and E.G. Karsten. By application 2723729/28-13. Declared Dec. 22, 1978. Publ. Sep. 23, 1980. BI 35).

Known in the art are methods of treating gastrointestinal hemorrhages that are based on the usage of endoscopic methods, clipping, ligation, injection of sclerosants or vasoactive drugs into the mucosal layer, and also coagulation using thermal and electrical methods (Anjiki H., Kamisawa T., Sanaka M., Ishii T., Kuyama Y. Endoscopic hemostasis techniques for upper gastrointestinal hemorrhage: A review. World J Gastrointest Endosc. 2010; 2 (2): 54-60). Furthermore, in case of bleeding from a putrescent tumor, necrotizing esophagitis or nonspecific erosive colitis, i.e. in case when the diffuse mucosa bleeding occurs without an obvious source, such known methods remain ineffective, while they also do not allow for objective control of stability of hemostasis.

Known in the art is a method of diagnosing aneurismal dilatations of vessel by administering intraluminal endovascular catheters with or without stents, involving guiding the catheter under control of a fluoroscopy and obturating an aneurism with a stent (Roszelle B.N., Nair P., Gonzalez L.F., Haithem Babiker M., Ryan J., Frakes D. Comparison among different high porosity stent configurations: hemodynamic effects of treatment in a large cerebral aneurysm. J Biomech Eng. 2014 February; 136 (2): 021013). However, if this known method is implemented, during the positioning of a catheter in a correct manner and identifying a defect, a circumferential blood flow may suffer, and hemorrhage from an aneurysm cavity may continue. Therefore existing analogues differ from the the claimed technique.

Known in the art is a method for detecting injuries of hollow organs by administering various coloring or radiopaque substance into a hollow organ (Ozimok C.J., Mellnick V.M., Patlas M.N. An international survey to assess use of oral and rectal contrast in CT protocols for penetrating torso trauma. Emerg Radiol. 2019 April; 26 (2): 117-121; Broder J.S., Hamedani A.G., Liu S.W., Emerman C.L. Emergency department contrast practices for abdominal/pelvic computed tomography-a national survey and comparison with the american college of radiology appropriateness criteria. J Emerg Med. 2013 February; 44 (2): 423-433). However, the manipulation itself only allows an injury to be ascertained, wherein this is not always possible since the success of ascertaining an injury strongly depends on the location of an injury and specifics of administration of a substance. In other words, this known method does not always allow for an injury of a hollow organ to be accurately located and does not allow for treatment of this injury.

### Gastrointestinal Bleedings

It should also be noted that gastrointestinal hemorrhages manifest themselves in the form of blood entering the lumen with subsequent exit through natural orifices evidenced by a characteristic clinical laboratory symptom complex. According to the recommendations of the Russian Society of Surgeons, hemorrhage control includes a set of treatment measures which can be conventionally divided into two groups: the first group of treatment measures affecting the hemostasis system activating coagulation, and the second group of treatment measures which is a pool of invasive manipulations directly affecting the source of hemorrhage. The first group of treatment measures involves a strict list of means and the order of their application, and the second group of treatment measures is variable to a large extent. A key principle of invasive techniques is the physical action on the source of hemorrhage (Ghassemi K.A., Jensen D.M. Evolving techniques for gastrointestinal endoscopic hemostasis treatment. Expert Rev Gastroenterol Hepatol. 2016; 10(5):615-623. doi: 10.1586/17474124.2016.1130623). In the case where a bleeding vessel or vein can be detected, a large number of techniques is available for stopping the hemorrhage (e.g., clipping, ligation, sclerosis, argon plasma coagulation, and/or the like). In the case of diffuse hemorrhage (e.g., bleeding from a tumor, nonspecific ulcerative colitis, foci of Crohn's disease, hemocolitis, etc.), methods for hemorrhage control are nonspecific and are often aimed at creating optimal conditions for hemorrhage control: the use of argon plasma endoscopic coagulation, submucosal administration of vasoconstrictor drugs, distribution of hemostatic drugs over the surface, or the like.

In particular, a method for argon plasma coagulation is known in the art which is a method for physical high-temperature action on a source of hemorrhage. This method for argon plasma coagulation is most often used for ulcerative sources in the stomach and duodenum, reducing the risk of complications by a factor of 3.5 (Economic efficiency of the endoscopic argon plasma coagulation method in ulcerative gastroduodenal hemorrhage/Mashkin A.M., Efanov A.V., Maleev A. V. et al. //Ural Medical Science and Education, 2015, 16((1)(81)): 95-98). In addition, the authors note that in the case of a diffuse lesion of the gastric and/or duodenal mucosa, the method for argon plasma coagulation is often ineffective, and a combination of various methods is required to achieve hemostasis, improving the desired effect up to 98% (Povalyaev A.V. Erosive gastrointestinal hemorrhage in risk group patients: clinical findings, diagnosis, treatment/Povalyaev A. V., Duvansky V.A., Chernekhovskaya N.E. //Bulletin of the N.I. Pirogov National Medical and Surgical Center 2020, 15(4): 39-44, DOI 10.25881/BPNMSC.2020.54.37.008; Shabunin A.V., Nechipay A.M., Korzheva I. Yu., Combined endoscopic hemostasis with the use of EndoClot in patients with gastroduodenal hemorrhage of ulcerative etiology /Annals of surgery, 2016; 21(6): 49-55).

Furthermore, a method for endoscopic clipping or endoclipping is known in the art, the method based on mechanical compression of the hemorrhage source with a special clip, the process essentially comparable to stitching of the hemorrhage site (Sung J.J., Tsoi K.K., Lai L.H., et al. Endoscopic clipping versus injection and thermo-coagulation in the treatment of non-variceal upper gastrointestinal bleeding: A metaanalysis. Gut. 2007; 56(10):1364-1373. Jensen D.M., Machicado G.A., Hirabayashi K. Randomized controlled study of 3 different types of hemoclips for hemostasis of bleeding canine acute gastric ulcers. Gastrointest Endosc. 2006; 64(5):768-773). The disadvantage of such a method for endoscopic clipping or endoclipping is the need to directly visualize the source of hemorrhage, and the result of utilizing said prior art method often depends on the anatomical features of the patient.

In addition, a method for injection-induced hemostasis or sclerotherapy is known, the method including administering a sub-mucosal solution of adrenaline or a special ethyl alcohol-based sclerosant (Marmo R., Rotondano G., Piscopo R., et al. Dual therapy versus monotherapy in the endoscopic treatment of high-risk bleeding ulcers: A meta-analysis of controlled trials. Am J Gastroenterol. 2007; 102(2):279-289; Vergara M., Bennett C., Calvet X., et al. Epinephrine injection versus epinephrine injection and a second method in high-risk bleeding ulcers. Cochrane Database Syst Rev. 2014; 10:CD005584.). The advantage of this method of injection-induced hemostasis or sclerotherapy is the non-targeted administration of the drug around the source of hemorrhage, wherein, due to moderate mechanical pressure and moderate impact on the vessels, the risk of spasm or obliteration is reduced. However, when using said method for injection-induced hemostasis or sclerotherapy, there exists a risk of injury to larger vessels or risk of perforation of a gastrointestinal organ during needle insertion into the wall of said organ. Such a method of injection-induced hemostasis or sclerotherapy is often used in combination with other hemostatic methods, since bypass surgery and intramural vascular anastomoses lead to a high risk of repeated hemorrhage.

In addition, the use of argon plasma coagulation for tumor recanalization in esophageal cancer is known in the art (Conservative treatment of locally advanced and inoperable esophageal cancer: opportunities and prospects/Kanaev S.V., Shcherbakov A.M., Tyuryaeva E.I., Avanesyan A.A. /Issues of oncology, 2012; 58(2):199-202), but the source does not disclose the use of argon plasma coagulation in the case of tumor disintegration and hemorrhage.

Further, a method for endoscopic application of polysaccharide-based hemostatic powder is known in the art (Kevin A. Ghassemi & Dennis M. Jensen (2016): Evolving techniques for gastrointestinal endoscopic hemostasis treatment, Expert Review of Gastroenterology & Hepatology, DOI: 10.1586/17474124.2016.1130623). The application of the method for endoscopic application of polysaccharide-based hemostatic powder leads to the formation of a barrier clot and to adhesion on the hemorrhaging surface, which in 85% of cases leads to a positive effect (Smith L.A., Stanley A.J., Bergman J.J., et al. Hemospray application in nonvariceal upper gastrointestinal bleeding: results of the Survey to Evaluate the Application of Hemospray in the Luminal Tract. J Clin Gastroenterol. 2014;48(10): e89-e92). However, in at least 15% of cases, repeated hemorrhage can be observed during monotherapy due to migration of the clot with the hemostatic powder caused by the peristalsis of the esophagus, stomach and intestine (Ibrahim M., El-Mikkawy A., Mostafa I., et al. Endoscopic treatment of acute variceal hemorrhage by using hemostatic powder TC-325: a prospective pilot study. Gastrointest Endosc. 2013; 78(5): 769-773).

It should be noted that the use of catheters for mechanical compression of the source of hemorrhage has been utilized since the 1950s, with the most common being the Sengstaken-Blakemore tube and the Milwaukee analogue thereof. Such prior art catheters are based on mechanical compression of submucosal vessels and obturation of the source of hemorrhage itself, with the indication for the use of such prior art catheters being a hemorrhage from varicose veins of the esophagus, the cardioesophageal junction and the stomach bottom. Most experts and authors agree that this method for hemorrhage treatment based on the use of catheters for mechanical compression of the source of hemorrhage is temporary with a duration of no more than 12 hours, while the failure rate thereof remains at around 10-15% (Yoshida H., Mamada Y., Taniai N., Yoshioka M., Hirakata A., Kawano Y., Mizuguchi Y., Shimizu T., Ueda J., Uchida E. Treatment modalities for bleeding esophagogastric varices. J Nippon Med Sch. 2012; 79(1):19-30. doi: 10.1272/jnms.79.19). Furthermore, the disadvantages of the method for hemorrhage treatment based on the use of catheters for mechanical compression of the source of hemorrhage can include the problem of placement of such catheters in the case of diaphragmatic hernias, as well as the development of necrotic changes in the mucosa of the esophagus caused by mechanical compression of the vessels.

### Gastrointestinal Fistulas

Gastrointestinal fistulas are severe complications with high mortality rate currently remaining at 5-20% (Vodyasov A. V., Kopaliani D.M., Yartsev P.A., Kaloeva O.K. Conservative treatment of patients with small bowel fistula. Khirurgiia (Mosk). 2021; (4):78-84. Russian. doi: 10.17116/hirurgia202104178). In the process of a defect in the gastrointestinal organ wall developing against the background of an inflammatory reaction and/or after traumatic exposure (e.g., trauma, surgery, parasitogenic effect, etc.), a perforation develops in said organ wall, through which the contents of said organ begin to enter the spaces and cavities surrounding said organ. The treatment process is complicated by the fact that due to inflammatory changes it is impossible to close the wound simply by suturing, obturing, swabbing or sealing the affected organ wall. The healing of the defect in the gastrointestinal organ wall occurs over a long period of time and is dependent on the extant wound infection process supported by an inflammatory reaction, as well as the loss of nutritional components, microelements and the issue of their replenishment; therefore, the search for means to solve the problem of the healing of defects in the gastrointestinal organ wall remains an issue of concern.

In particular, a method for stenting a cavity of a damaged organ with a covered self-expanding or plastic stent is known in the art, wherein the wall defect is closed, and the cavity of the organ is separated from the surrounding area, which prevents the ingress of organ content and induces healing. According to statistics, a successful outcome is usually observed only in 53-67% of cases, which is associated with an increase in inflammation of the originally infected wound in the case of insufficient drainage. In a number of cases and due to a number of reasons (including the anatomical features of the patient, errors or deviations in the prescribed diet, ongoing inflammation, and/or the like), the stent migrates, which can lead to both the opening of the fistulous tract and an increase in the primary defect (Brangewitz M., Voigtländer T., Helfritz F.A., Lankisch T.O., Winkler M., Klempnauer J., Manns M.P., Schneider A.S., Wedemeyer J. Endoscopic closure of esophageal intrathoracic leaks: stent versus endoscopic vacuum-assisted closure, a retrospective analysis. Endoscopy. 2013; 45(6):433-438. doi: 10.1055/s-0032-1326435; Tuebergen D., Rijcken E., Mennigen R. et al. Treatment of thoracic esopha-geal anastomotic leaks and esophageal perforations with endoluminal stents: efficacy and current limitations. J Gastrointest Surg 2008; 12:1168-1176).

Further, a vacuum therapy method for treating gastrointestinal wall defects is known in the art. The use of the vacuum therapy method in the treatment of gastrointestinal wall defects ensures the continuous removal of intestinal content and pus from the abdominal cavity (Wild T., Stortecky A., Stremitzer S., Lechner P., Humpel G., Glaser K., Fortelny R., Karner J., Sautner T. Abdominal dressing—a new standard in therapy of the open abdomen following secondary peritonitis? Zentralbl Chir 2006; 131: S111-S114. In German). Continuous sanitization of the abdominal cavity leads to the situation wherein, due to secondary reparative processes, the wound surface is separated from the cavity, the absorption of degradation products through the peritoneum decreases, and gradual complete healing, or the formation of a complete external fistula, occurs. In the case of the complete external fistula, the second stage of surgical treatment begins after six (6) months. Nevertheless, the effectiveness of the above approach remains at 60-80%, since, on the one hand, the effectiveness of treatment largely depends on the recovery capabilities of the patient's body, and on the other hand, the vacuum therapy itself can be the cause of new fistulas and perforations forming (Wild T., Goetzinger P., Telekey B. VAC and fistula formation. Colorectal Dis. 2007 July; 9(6):572-3. doi: 10.1111/j.1463-1318.2007.01309.x.).

Furthermore, an improved endoscopic vacuum therapy (EVAC) method is known in the art, the method based on the same principle of limiting or stopping the flow of gastrointestinal content into the surrounding cavities and/or spaces, followed by sanitization from purulent cavities (Brangewitz M., Voigtländer T., Helfritz F.A., Lankisch T.O., Winkler M., Klempnauer J., Manns M. P, Schneider A.S., Wedemeyer J. Endoscopic closure of esophageal intrathoracic leaks: stent versus endoscopic vacuum-assisted closure, a retrospective analysis. Endoscopy. 2013; 45(6):433-438. doi: 10.1055/s-0032-1326435). The positive outcome of the gastrointestinal organ defect treatment with EVAC therapy is observed in 80% of the cases, which is generally due to the following two issues. Firstly, the aspirating sponge is regularly clogged with fibrin and the gastrointestinal content, and therefore ceases to function. Secondly, the patient has to be transferred to parenteral nutrition, which is often not fully effective, while the absence of nutrient media in the esophagus and the intestine causes wall damage.

In addition, a method for flow-through flushing treatment of fistula is known in the art, the method being one of the varieties of EVAC therapy (Vodyasov A. V., Kopaliani D.M., Yartsev P.A., Kaloeva O.K. Conservative treatment of patients with small bowel fistula. Khirurgiia (Mosk). 2021; (4):78-84. Russian. doi: 10.17116/hirurgia202104178). This method for flow-through flushing treatment of fistula includes various combinations of conducting and discharging drains, wherein a solution, including an antibacterial solution, is supplied through the drains of the first type, and the previously supplied solution is drained along with pus and organ content through the drains of the second type, thus ensuring removal of said liquid mixture into special containers. One of the main disadvantages of this method for flow-through flushing treatment is that it is not always possible to establish an adequate outflow of content. Due to the organs of the abdominal cavity (greater omentum, intestinal loops, mesentery, etc.), drains are separated, which leads to poor drainage with the subsequent development of abscesses. The continuous loss of intestinal content removed by the drains leads to the loss of proteins, carbohydrates, fats and microelements, and thus the problem of providing nutritional support remains unresolved. The continuous intake of both pus and intestinal content leads to prolonged peritonitis and absorption of inflammation products by the peritoneum, which leads to the development of both a systemic inflammatory response and multiple organ failure.

### Acute Pancreatitis

Acute pancreatitis is an inflammatory autolytic process that develops due to activation of enzymes in the cells, ducts and intercellular space of the mammalian pancreas, and, simultaneously with said autolytic process, the systemic inflammatory response syndrome usually develops, further leading to the development of multiple organ failure. According to the international recommendations of 2013 and the European recommendations of 2019 for the treatment of acute pancreatitis, the treatment of acute pancreatitis includes active infusion therapy and correction of complications developing as a result of pancreatic inflammation (Working Group IAP/APA Acute Pancreatitis Guidelines. IAP/APA evidence-based guidelines for the management of acute pancreatitis. Pancreatology. 2013 July-August; 13(4 Suppl 2): e1-15. doi: 10.1016/j.pan.2013.07.063; Leppäniemi, A., Tolonen, M., Tarasconi, A. et al. 2019 WSES guidelines for the management of severe acute pancreatitis. World J Emerg Surg 14, 27. https://doi.org/10.1186/s13017-019-0247-0).

The topographic anatomical position of the pancreas makes the organ very difficult to access for manipulation and treatment. When inflammation develops, it is often non-specific, and complications extend to surrounding organs and spaces.

The main method for treatment of acute pancreatitis, recommended both in the above international recommendations from 2013 and European recommendations from 2019 for the treatment of acute pancreatitis, and by the Russian Society of Surgeons (Acute pancreatitis: Clinical recommendations/Dibirov M.D., Bagnenko S.F., Blagovestnov D.A. et al.; Russian Society of Surgeons, Association of Hepatopancreatobiliary Surgeons of the CIS Moscow: Ministry of Health of the Russian Federation, 2015-pp. 38), is the method for treating acute pancreatitis with active infusion therapy. It should be noted that one of the disadvantages of said method for treatment of acute pancreatitis with active infusion therapy is that the inflammation itself is only impacted indirectly, while the key impact is caused on various pathological processes, the correction of which is carried out by active washout of inflammation complexes.

Patent of the Russian Federation No. 2607929 (hereinafter referred to as RU 2607929), filed on Jan. 10, 2017, discloses a device for treatment of pancreatitis, the device implementing the method for treatment of acute pancreatitis by improving the outflow of pancreatic juice and bile using controlled negative pressure (vacuum). Due to the fact that during the first few days of acute pancreatitis, the duodenal peristalsis is disrupted, the active aspiration created by the device of RU 2607929 restores the physiological outflow of secreted fluid from organs. The disadvantage of the method for treatment of acute pancreatitis of RU 2607929 lies in the design features of the device of RU 2607929 used to implement the method. Specifically, the device for treatment of pancreatitis of RU 2607929 comprises a cylinder-shaped device providing optimal fitting with the duodenal wall and forming a cavity around the major duodenal papilla. Given the variation in anatomical forms of pancreatic duct inflow (with single outflow duct or with an additional) and the possible presence of parapapapillary diverticulum, the device for treatment of pancreatitis of RU 2607929 may not be applicable to a number of patients, and may further cause obturation and therefore even cause pancreatitis. The device for treatment of pancreatitis of RU 2607929, when inserted through the nose, can lead to trauma to the nasal passages, and when placed in the duodenum, can assume an uncomfortable position, in particular, the device for attachment to the duodenum wall can assume a position that is not opposite with respect to the duodenal papilla, thus potentially requiring a separate complex endoscopic manipulation to rotate said device to ensure its placement opposite the duodenal papilla.

Furthermore, U.S. Pat. No. 8,529,612 (hereinafter referred to as U.S. Pat. No. 8,529,612), filed on Feb. 4, 2010, discloses a method for treatment of acute pancreatitis, including severe form thereof, by providing a local cooling region. Thus, the method for treatment of acute pancreatitis disclosed in U.S. Pat. No. 8,529,612 is based on artificially created hypothermia inhibiting various proteolytic enzymes and the production of inflammatory factors, which reduces the activity of systemic inflammatory response syndrome (SIRS) or even halts the development of said syndrome. When implementing the method for treatment of acute pancreatitis disclosed in U.S. Pat. No. 8,529,612, hypothermia is created by inserting a catheter with one or more balloons, wherein the cooled liquid is fed into said balloons or replaced therein using a pump. Further, when implementing the method for treatment of acute pancreatitis described in U.S. Pat. No. 8,529,612, the exchange rate of the cooled liquid is controlled either by varying the rate of supply and withdrawal of this cooled liquid, or automatically by using a solution temperature sensor detecting the attainment of a set exchange rate value for the cooled liquid, at which point the pump is switched on and the liquid in the balloons is replaced. It should be noted that the method for treatment of acute pancreatitis described in U.S. Pat. No. 8,529,612 is a modification of a prior art standard treatment method for pancreatitis based on applying an ice pack to the anterior abdominal wall. An advantage of the method for treatment of acute pancreatitis disclosed in U.S. Pat. No. 8,529,612 is that the cooling region formed is located in close proximity to the pancreas, which eliminates the influence of skin, subcutaneous tissue and other organs on the process of cooling the retroperitoneal space. The disadvantage of said prior art method for treatment of acute pancreatitis disclosed in U.S. Pat. No. 8,529,612 is that the inventors of U.S. Pat. No. 8,529,612 propose installing fluid bottles in the stomach of a patient in an isolated manner for an extended period of time, including in combination with insertion into the duodenum and the jejunum. In particular, if the patient has intestinal paresis in the acute phase of pancreatitis and parapancreatic infiltrate, the introduction of liquid balloons weighing over 300 g would lead to unpleasant sensation in the patient body (e.g., abdominal pain, heavy feeling, nausea, vomiting), especially when filled with liquid balloons installed in different parts of the intestine. It should be noted that prolonged exposure of the wall to liquid balloons weighing over 100 g often leads to ischemic changes in the gastric mucosa. The authors of U.S. Pat. No. 8,529,612 refer to the experience of using balloons for the treatment of obesity, however, in the case of acute pancreatitis, a variety of prior art information sources indicates the presence of microthromboses and changes in microcirculation in the organs of said anatomical region in acute pancreatitis cases (Kroner P.T., Wallace M.B., Raimondo M., Antwi S.O., Ma Y., Li Z., Ji B., Bi Y. Systemic anticoagulation is associated with reduced mortality and morbidity in acute pancreatitis. Pancreatology. 2021 December; 21(8): 1428-1433. doi: 10.1016/j.pan.2021.09.003). It should be apparent to those skilled in the art that, in implementing the method for treatment of acute pancreatitis disclosed in U.S. Pat. No. 8,529,612, balloon extension would result in developing obturation of the gastric, duodenal, and jejunal lumen; however, the inventors have not proposed any solution to this problem at all in U.S. Pat. No. 8,529,612, in particular, they have not described the process for carrying out drainage of various parts of the gastric tract. Thus, the catheter in various variations thereof according to the method for treatment of acute pancreatitis disclosed in U.S. Pat. No. 8,529,612 cannot remain in the body for more than 6 hours after placement, as otherwise the patient can develop a complication of acute mechanical obstructive intestinal obstruction, which is a very serious adverse condition. Furthermore, U.S. Pat. No. 8,529,612 does not address the issue of preventing catheter migration and preventing obturation of the major duodenal papilla, which could lead to a worsening of the course of acute pancreatitis.

Further, methods for treatment of acute pancreatitis by administering various drugs to reverse regulation of pancreatic secretory function are known in the art, the drugs including, e.g., choline blocking drugs (Savelyev V.S. et al. Acute pancreatitis—M.: Medicine, 1983; Chaplinsky V.V., Gnatyshak A.I. Acute pancreatitis—M.: Medicine, 1972), protease inhibitors (Filin V.I., Kostyuchenko A.L. Emergency pancreatology—St. Petersburg, 1994, Sokolov V.I. Surgical diseases of the pancreas—M.: Medicine, 1998), dalargin (Filin V.I., Kostyuchenko A.L. Emergency pancreatology—St. Petersburg, 1994), somatotropic hormone analogues (Filin V.I., Kostyuchenko A.L. Emergency pancreatology.—St. Petersburg, 1994; Shaposhnikov A.V. Russian Journal of Gastroenterology, Hepatology, Coloproctology—1996, 4: 85-89; Uhl W. et al. Gut.—1999.—Vol. 40.-pp. 424-429), trypsin (RU 2198680 patent for the invention titled “A method for treatment of acute pancreatitis”, filed on February 20, 2003). According to the prior art information sources, acute pancreatitis causes the development of pathological hyperstimulation of the excretory function of the pancreas, and thus the introduction of a number of drugs, including locally into the duodenum, is aimed at reducing enzyme production. Analysis of data from prior art information sources showed that the use of any of the above agents did not significantly improve the course of acute pancreatitis and was not included in any of the standard recommendations for the treatment of the disease (except for the growth hormone analogues), which, in the opinion of the authors of the present application, is associated with both the toxicity of a number of drugs and their insufficient impact on all pathophysiological chains of autolytic inflammation.

In addition, Russian Patent No. 2263517 (hereinafter referred to as RU 2263517), filed on Nov. 10, 2005, discloses a method for treatment and prevention of acute pancreatitis by stenting the pancreatic duct. The method for treatment and prevention of acute pancreatitis disclosed in RU 2263517 is most commonly used in the treatment of biliary pancreatitis, in particular, for preventing the development of pancreatitis after mechanical trauma, e.g., after cholangiopancreatography. Approaches to the treatment and prevention of pancreatitis similar to the method for treatment and prevention of acute pancreatitis disclosed in RU 2263517 have been introduced into the standard recommendations as a measure for prevention of pancreatitis after various interventions in the ductal system, and they are based on ensuring the timely outflow of pancreatic juice from the gland in order to prevent enzyme activation in the organ. However, at present, the use of such approaches in the case of acute pancreatitis is very limited, since against the background of major duodenal papilla dysfunction and impaired duodenal motility, the use of such approaches often leads to the introduction of intestinal content into the ductal system and an increase in the level of pancreatic hypertension, which in turn can lead to a worsening of the course of acute pancreatitis.

### Small Intestine Wall Lesions

When hollow organs of the abdominal cavity are damaged or injured by bodies of high kinetic energy, several areas are formed in the wall of said hollow organs, including the necrotic zone and the zone of molecular contusion. The ultimate volume of the abdominal hollow organ lesion is formed 24-72 hours after sustaining the injury. In primary care, it is not possible to predict and estimate the ultimate extent of the hollow organ lesion in the first hours after sustaining the injury. It is not feasible to leave the defects or lesions of the gastrointestinal tract uncovered, as this will lead to peritonitis development and to worsening of the course of the disease. Common approaches to treatment of defects or lesions in the walls of injured abdominal hollow organs are simple suturing of such lesions in the walls of injured organs, primary resection of damaged or injured intestine loops, or a combination of such approaches. As one of the approaches to the treatment of such injuries, suturing of lesions in the walls of injured abdominal organs takes less time and requires a less qualified specialist, solving the issue of peritonitis development, but in most cases, when using this approach, the development of repeated perforations occurs after 48-96 hours in the suturing area due to the ultimate formation of the necrotic zone (zone of necrosis). The suturing of lesions in the walls of injured abdominal organs leads to lengthy treatment of the patient taking up several months, high mortality rate and a high risk of gastrointestinal fistula formation, requiring prolonged rehabilitation and high-tech medical care. In the case of primary resection of injured intestine loops, the risk remains that an area of the intestine that has suffered a peripheral injury will be left untreated, and that an area of necrosis may further develop after a certain period of time. Another issue with primary resection of injured intestine loops is excessive removal of intestine loops which can lead to malabsorption or maldigestion, and lifelong nutritional ailments for the patient.

A separate surgical problem is the injury to the duodenum and the ligament of Treitz anatomical region (the junction between the duodenum and the initial jejunum). Due to anatomical location specifics, in such cases it is impossible to perform a standard resection, and the presence of an extremely aggressive media formed by a mixture of pancreatic juice, bile and gastric juice is the reason for the development of dehiscence, fistula and high mortality rate among patients.

Organizationally, the above types of wounds or lesions in the hollow organs of the abdominal cavity of patients require both the presence of individual abdominal surgeons and the large amount of time required to provide primary specialized medical care. On average, any of the two above approaches to treatment of defects or lesions in the walls of injured hollow abdominal organs takes up 1.5-3 hours which, in the face of a large number of incoming injured patients, can lead to late administration of medical care and, consequently, to an increase in the likelihood of fatalities.

One of illustrative treatment methods based on the use of a similar catheter for isolating an interior of a mammal hollow organ is disclosed in U.S. Pat. No. 9,526,874 published on 30 Jun. 2015. In particular, the catheter disclosed in U.S. Pat. No. 9,526,874 comprises an elongate body designed to be inserted into a lumen of a human hollow organ and two balloons configured to be inflated to isolate an interior of the hollow organ therebetween; and a functional channel extending in the body and comprising a functional opening provided in the body between the balloons, wherein the functional channel is designed to allow: a negative pressure to be produced in the isolated interior to take therefrom a biological fluid being specific for the hollow organ or a liquid or gaseous medium to be supplied into the isolated interior.

Other treatment methods based on the use of similar catheters for isolating an interior of a mammal hollow organ are disclosed in WO 2021/137739 (hereinafter WO 2021/137739) published on 1 Nov. 1988, U.S. Pat. No. 4,781,677 (hereinafter U.S. Pat. No. 4,781,677) published on 1 Nov. 1988 and U.S. Pat. No. 5,951,514 published on 14 Sep. 1999.

A main disadvantage of treatment methods based on the use of the catheter disclosed in U.S. Pat. No. 9,526,874 or other prior art documents is in that the catheter cannot be inserted into a lumen of a mammal hollow organ for a long time due to the lack of physiological connectivity between hollow organ sections adjoining the hollow organ region isolated by the inflated balloons and being outside the isolated region of the mammal hollow organ, in particular the catheter needs to be used for treatment of an esophageal or intestinal wall injury, treatment of bleeding into an esophageal or intestinal lumen, treatment of acute pancreatitis.

Therefore, treatment methods based on the use of catheters for isolating an interior of a mammal hollow organ are to be further developed, in particular to allow the use of such catheters for a long time within mammal hollow organs for treatment of an esophageal or intestinal wall injury, treatment of bleeding into an esophageal or intestinal lumen, treatment of acute pancreatitis. Consequently, a technical problem to be solved by the present invention is to develop treatment methods based on the use of a catheter for isolating an interior of a mammal hollow organ that would at least partly eliminate the above disadvantage of the prior art treatment methods, i.e. to eliminate the problem of the lack of possibility to insert a catheter into a lumen of a mammal hollow organ and maintain the inserted catheter therein for a long time while maintaining a function of the mammal hollow organ, in particular when using the catheter for treatment of an esophageal or intestinal wall injury, treatment of bleeding into an esophageal or intestinal lumen or treatment of acute pancreatitis.

### SUMMARY OF INVENTION

An objective of the present invention is to develop treatment methods based on the use of a catheter for isolating an interior of a mammal hollow organ, each treatment method solving at least the above technical problem.

To achieve the objective of the invention, as embodied and broadly described herein, in a first aspect of the present invention, there is provided a method for treatment of an esophageal or intestinal wall injury, the method comprising:
(i) inserting a catheter (100) for isolating an interior of a mammal hollow organ into a mammal esophagus or mammal intestine, the catheter comprising:
an elongate body designed to be inserted into a lumen of the mammal hollow organ;
two balloons disposed along the catheter body and configured to be inflated to isolate an interior of the mammal hollow organ therebetween; and
a functional channel extending in the catheter body and comprising a functional opening provided in the catheter body between the balloons,
wherein the functional channel is designed to allow:
   a negative pressure to be produced in the isolated interior to take a fluid or gaseous medium therefrom via the functional opening; and/or
   a liquid or gaseous medium to be supplied into the isolated interior via the functional opening; and
   (ii) inflating the balloons (4) to isolate the interior of the mammal esophagus or mammal intestine therebetween; and
   (iii) producing, by means of the functional channel (3) of the catheter (100), a negative pressure in the isolated interior for taking a biological content being specific for the mammal esophagus or mammal intestine therefrom via the functional opening (7) and/or supplying, by means of the functional channel (3) of the catheter (100), a liquid or gaseous medium appropriate for treatment of the esophageal or intestinal wall injury to the isolated interior via the functional opening (7).

To achieve the objective of the invention, as embodied and broadly described herein, in a second aspect of the present invention, there is provided a method for treatment of an esophageal or intestinal wall injury, the method comprising:
(ii) inserting a catheter (100) for isolating an interior of a mammal hollow organ into a mammal esophagus or mammal intestine, catheter comprising:
an elongate body designed to be inserted into a lumen of the mammal hollow organ;
two balloons disposed along the catheter body and configured to be inflated to isolate an interior of the mammal hollow organ therebetween; and
a functional channel extending in the catheter body and comprising a functional opening provided in the catheter body between the balloons,
wherein the functional channel is designed to allow:
   a negative pressure to be produced in the isolated interior to take a fluid or gaseous medium therefrom via the functional opening; and/or
   a liquid or gaseous medium to be supplied into the isolated interior via the functional opening; and
wherein the catheter body is further provided with a net or enclosure being permeable to a fluid and/or gaseous medium, wherein the net or enclosure at least partly encloses a catheter part defined by the balloons so as to cover the functional opening, and wherein the net or enclosure is attached to the balloons or encloses them such that the net or enclosure becomes strained when the balloons are inflated,
(ii) inflating the balloons (4) to isolate the interior of the mammal esophagus or mammal intestine therebetween; and
(iii) producing, by means of the functional channel (3) of the catheter (100), a negative pressure in the isolated interior for taking a biological content being specific for the mammal esophagus or mammal intestine therefrom via the functional opening (7) and/or supplying, by means of the functional channel (3) of the catheter (100), a liquid or gaseous medium appropriate for treatment of the esophageal or intestinal wall injury to the isolated interior via the functional opening (7).

To achieve the objective of the invention, as embodied and broadly described herein, in a third aspect of the present invention, there is provided a method for treatment of an esophageal or intestinal wall injury, the method comprising:
(i) inserting a catheter for isolating an interior of a mammal hollow organ, the catheter comprising:
an elongate body designed to be inserted into a lumen of the mammal hollow organ;
two balloons disposed along the catheter body and configured to be inflated to isolate the interior of the mammal hollow organ therebetween; and
a functional channel extending in the catheter body and comprising a functional opening provided in the catheter body between the balloons,
wherein the functional channel is designed to allow:
   a negative pressure to be produced in the isolated interior to take a fluid or gaseous medium therefrom via the functional opening; and/or
   a liquid or gaseous medium to be supplied into the isolated interior via the functional opening; and
wherein the functional opening is positioned between two ring-shaped enclosing projections provided on the catheter body between the balloons,
(ii) inflating the balloons (4) to isolate the interior of the mammal esophagus or mammal intestine therebetween; and
(iii) producing, by means of the functional channel (3) of the catheter (100), a negative pressure in the isolated interior for taking a biological content being specific for the mammal esophagus or mammal intestine therefrom via the functional opening (7) and/or supplying, by means of the functional channel (3) of the catheter (100), a liquid or gaseous medium appropriate for treatment of the esophageal or intestinal wall injury to the isolated interior via the functional opening (7).

To achieve the objective of the invention, as embodied and broadly described herein, in a fourth aspect of the present invention, there is provided a method for treatment of bleeding into an esophageal or intestinal lumen, the method comprising:
(i) inserting a catheter (100) for isolating an interior of a mammal hollow organ into a mammal esophagus or mammal intestine, the catheter comprising:
an elongate body designed to be inserted into a lumen of the mammal hollow organ;
two balloons (4) disposed along the catheter body and configured to be inflated to isolate the interior of the mammal hollow organ therebetween; and
a functional channel (3) extending in the catheter body and comprising a functional opening (7) provided in the catheter body between the balloons (4),
wherein the functional channel (3) is designed to allow:
   a negative pressure to be produced in the isolated interior to take a fluid or gaseous medium therefrom via the functional opening (7); and/or
   a liquid or gaseous medium to be supplied into the isolated interior via the functional opening (7); and
(ii) inflating the balloons (4) to isolate the interior of the mammal esophagus or mammal intestine therebetween; and
(iii) producing, by means of the functional channel (3) of the catheter (100), a negative pressure in the isolated interior for taking a biological content being specific for the mammal esophagus or mammal intestine therefrom via the functional opening (7) and/or supplying, by means of the functional channel (3) of the catheter (100), a liquid or gaseous medium appropriate for treatment of the bleeding into the esophageal or intestinal lumen to the isolated interior via the functional opening (7).

To achieve the objective of the invention, as embodied and broadly described herein, in a sixth aspect of the present invention, there is provided a method for treatment of bleeding into an esophageal or intestinal lumen, the method comprising:
(i) inserting a catheter (100) for isolating an interior of a mammal hollow organ into a mammal esophagus or mammal intestine, catheter comprising:
an elongate body designed to be inserted into a lumen of the mammal hollow organ;
two balloons (4) disposed along the catheter body and configured to be inflated to isolate the interior of the mammal hollow organ therebetween; and
a functional channel (3) extending in the catheter body and comprising a functional opening (7) provided in the catheter body between the balloons (4),
wherein the functional channel (3) is designed to allow:
   a negative pressure to be produced in the isolated interior to take a fluid or gaseous medium therefrom via the functional opening (7); and/or
   a liquid or gaseous medium to be supplied into the isolated interior via the functional opening (7); and
wherein the catheter body is further provided with a net or enclosure (16) being permeable to a fluid and/or gaseous medium, wherein the net or enclosure (16) at least partly encloses a catheter part defined by the balloons (4) so as to cover the functional opening (7), and wherein the net or enclosure (16) is attached to the balloons (4) or encloses them such that the net or enclosure (16) becomes strained when the balloons (4) are inflated,
(ii) inflating the balloons (4) to isolate the interior of the mammal esophagus or mammal intestine therebetween; and
(iii) producing, by means of the functional channel (3) of the catheter (100), a negative pressure in the isolated interior for taking a biological content being specific for the mammal esophagus or mammal intestine therefrom via the functional opening (7) and/or supplying, by means of the functional channel (3) of the catheter (100), a liquid or gaseous medium appropriate for treatment of the bleeding into the esophageal or intestinal lumen to the isolated interior via the functional opening (7).

To achieve the objective of the invention, as embodied and broadly described herein, in a sixth aspect of the present invention, there is provided a method for treatment of bleeding into an esophageal or intestinal lumen, the method comprising:
(i) inserting a catheter (100) for isolating an interior of a mammal hollow organ into a mammal esophagus or mammal intestine, the catheter comprising:
an elongate body designed to be inserted into a lumen of the mammal hollow organ;
two balloons (4) disposed along the catheter body and configured to be inflated to isolate the interior of the mammal hollow organ therebetween; and
a functional channel (3) extending in the catheter body and comprising a functional opening (7) provided in the catheter body between the balloons (4),
wherein the functional channel (3) is designed to allow:
   a negative pressure to be produced in the isolated interior to take a fluid or gaseous medium therefrom via the functional opening (7); and/or
   a liquid or gaseous medium to be supplied into the isolated interior via the functional opening (7); and
wherein the functional opening (7) is positioned between two ring-shaped enclosing projections (6) provided on the catheter body between the balloons (4),
(ii) inflating the balloons (4) to isolate the interior of the mammal esophagus or mammal intestine therebetween; and
(iii) producing, by means of the functional channel (3) of the catheter (100), a negative pressure in the isolated interior for taking a biological content being specific for the mammal esophagus or mammal intestine therefrom via the functional opening (7) and/or supplying, by means of the functional channel (3) of the catheter (100), a liquid or gaseous medium appropriate for treatment of the bleeding into the esophageal or intestinal lumen to the isolated interior via the functional opening (7).

To achieve the objective of the invention, as embodied and broadly described herein, in a seventh aspect of the present invention, there is provided a method for treatment of acute pancreatitis, the method comprising:
(i) inserting a catheter (100) for isolating an interior of a mammal hollow organ into a mammal esophagus or mammal intestine, the catheter comprising:
an elongate body designed to be inserted into a lumen of the mammal hollow organ;
two balloons (4) disposed along the catheter body and configured to be inflated to isolate the interior of the mammal hollow organ therebetween; and
a functional channel (3) extending in the catheter body and comprising a functional opening (7) provided in the catheter body between the balloons (4),
wherein the functional channel (3) is designed to allow:
   a negative pressure to be produced in the isolated interior to take a fluid or gaseous medium therefrom via the functional opening (7); and/or
   a liquid or gaseous medium to be supplied into the isolated interior via the functional opening (7); and
(ii) inflating the balloons (4) to isolate the interior of the mammal esophagus or mammal intestine therebetween; and
(iii) producing, by means of the functional channel (3) of the catheter (100), a negative pressure in the isolated interior for taking a biological content being specific for the mammal esophagus or mammal intestine therefrom via the functional opening (7) and/or supplying, by means of the functional channel (3) of the catheter (100), a liquid or gaseous medium appropriate for treatment of the acute pancreatitis to the isolated interior via the functional opening (7).

To achieve the objective of the invention, as embodied and broadly described herein, in an eighth aspect of the present invention, there is provided a method for treatment of acute pancreatitis, the method comprising:
(i) inserting a catheter (100) for isolating an interior of a mammal hollow organ into a mammal esophagus or mammal intestine, catheter comprising:
an elongate body designed to be inserted into a lumen of the mammal hollow organ;
two balloons (4) disposed along the catheter body and configured to be inflated to isolate the interior of the mammal hollow organ therebetween; and
a functional channel (3) extending in the catheter body and comprising a functional opening (7) provided in the catheter body between the balloons (4),
wherein the functional channel (3) is designed to allow:
   a negative pressure to be produced in the isolated interior to take a fluid or gaseous medium therefrom via the functional opening (7); and/or
   a liquid or gaseous medium to be supplied into the isolated interior via the functional opening (7); and
wherein the catheter body is further provided with a net or enclosure (16) being permeable to a fluid and/or gaseous medium, wherein the net or enclosure (16) at least partly encloses a catheter part defined by the balloons (4) so as to cover the functional opening (7), and wherein the net or enclosure (16) is attached to the balloons (4) or encloses them such that the net or enclosure (16) becomes strained when the balloons (4) are inflated,
(ii) inflating the balloons (4) to isolate the interior of the mammal esophagus or mammal intestine therebetween; and
(iii) producing, by means of the functional channel (3) of the catheter (100), a negative pressure in the isolated interior for taking a biological content being specific for the mammal esophagus or mammal intestine therefrom via the functional opening (7) and/or supplying, by means of the functional channel (3) of the catheter (100), a liquid or gaseous medium appropriate for treatment of the acute pancreatitis to the isolated interior via the functional opening (7).

To achieve the objective of the invention, as embodied and broadly described herein, in a ninth aspect of the present invention, there is provided a method for treatment of acute pancreatitis, the method comprising:
(i) inserting a catheter (100) for isolating an interior of a mammal hollow organ into a mammal esophagus or mammal intestine, the catheter comprising:
an elongate body designed to be inserted into a lumen of the mammal hollow organ;
two balloons (4) disposed along the catheter body and configured to be inflated to isolate the interior of the mammal hollow organ therebetween; and
a functional channel (3) extending in the catheter body and comprising a functional opening (7) provided in the catheter body between the balloons (4),
wherein the functional channel (3) is designed to allow:
   a negative pressure to be produced in the isolated interior to take a fluid or gaseous medium therefrom via the functional opening (7); and/or
   a liquid or gaseous medium to be supplied into the isolated interior via the functional opening (7); and
wherein the functional opening (7) is positioned between two ring-shaped enclosing projections (6) provided on the catheter body between the balloons (4),
(ii) inflating the balloons (4) to isolate the interior of the mammal esophagus or mammal intestine therebetween; and
(iii) producing, by means of the functional channel (3) of the catheter (100), a negative pressure in the isolated interior for taking a biological content being specific for the mammal esophagus or mammal intestine therefrom via the functional opening (7) and/or supplying, by means of the functional channel (3) of the catheter (100), a liquid or gaseous medium appropriate for treatment of the acute pancreatitis to the isolated interior via the functional opening (7).

A main technical effect provided by the treatment method according to any of the first, second, third, fourth, fifth, sixth, seventh, eighth and ninth aspects of the present invention is prevented or excluded formation of congestive and/or inflammatory processes which would occur in the hollow organ due to the accumulation of mucus and other biological contents being specific for the hollow organ outside the isolated interior defined by the balloons. In particular, in the present invention, the formation of congestive and/or inflammatory processes in the hollow organ is prevented or excluded due to the fact that mucus and other biological contents being specific for the hollow organ, when accumulated in the hollow organ, may enter the inlet provided in the catheter body outside the isolated interior defined by the balloons, and may leave through the outlet provided in the catheter body outside the isolated interior defined by the balloons.

A further technical effect provided by the treatment method according to any of the second, fifth and eighth aspects of the present invention is reduced lesion or improved safety of the treatment due to prevented suction of a tissue of the hollow organ into the functional opening of the catheter body that would result in damage to the tissue. In particular, the prevented suction of the tissue of the hollow organ into the functional opening and, therefore, reduced lesion or improved safety of the treatment is provided due to the fact that the functional opening is covered by the net or enclosure, and the net or enclosure becomes strained when the balloons are inflated.

Another technical effect provided by the treatment method according to any of the second, fifth and eighth aspects of the present invention is increased reliability of the treatment due to prevented partial or complete blockage or occlusion of the functional opening that may be caused by suction of the tissue of the hollow organ into the functional opening and that would result in necessity to retrieve the mounted catheter from the hollow organ of the mammal and, then, to change the retrieved catheter to a new catheter or remove the hollow organ tissue entered into the functional opening from the functional opening of the retrieved catheter.

The treatment method according to any of the third, sixth and ninth aspects of the present invention also provides the above further technical effect which is reduced lesion or improved safety of the treatment due to the further prevented suction of a tissue of the hollow organ into the functional opening that would result in damage to the tissue. In particular, suction of the hollow organ tissue into the functional opening is further prevented due to the fact that the projections enclose the functional opening provided in the catheter body; enclosing projections do not allow the functional opening to contact directly to the hollow organ tissue or approach the hollow organ tissue for a distance appropriate for suction of the tissue into the functional opening when the negative pressure is produced in the isolated interior defined by the balloons.

The treatment method according to any of the third, sixth and ninth aspects of the present invention also provides the above another technical effect which is increased reliability of the treatment due to further prevented partial or complete blockage or occlusion of the functional opening that may be caused by suction of the tissue of the hollow organ into the functional opening and that would result in necessity to retrieve the mounted catheter from the hollow organ of the mammal and, then, to change the retrieved catheter to a new catheter or remove the hollow organ tissue entered into the functional opening from the functional opening of the retrieved catheter. In particular, the partial or complete blockage or occlusion of the functional opening is further prevented due to the fact that the projections enclose the functional opening provided in the catheter body.

The above-mentioned further technical effect, namely the reduced lesion or improved safety of the treatment, is further provided by the treatment method according to any of the third, sixth and ninth aspects of the present invention due to the fact that the projections enclosing the functional opening positioned therebetween are provided between the balloons, and the enclosing projections are ring-shaped. In particular, the reduced lesion or improved safety of the treatment is further provided due to extension or increase of a volume of the isolated interior in the hollow organ of the mammal, the isolated interior being used for producing the negative pressure therein; it allows the possibilities of production of the controlled negative pressure in the isolated interior of the hollow organ to be essentially expanded, the negative pressure having a level required to provide the suction and being specific for different hollow organs of mammals with different physiological features of such hollow organs, thereby preventing the excessive negative pressure from being produced in the isolated interior that would result in suction of a tissue of the hollow organ into the functional opening and, consequently, would result in damage to the tissue.

The above-mentioned another technical effect, namely the increased reliability of the treatment, is further provided by the treatment method according to any of the third, sixth and ninth aspects of the present invention due to the fact that the projections enclosing the functional opening positioned therebetween are provided between the balloons, and the enclosing projections are ring-shaped. In particular, the increased reliability of the treatment is further provided by the treatment method according to any of the third, sixth and ninth aspects of the present invention due to the fact that increased volume of the isolated interior where the negative pressure may be produced allows the possibilities of production of the controlled negative pressure in the isolated interior of the hollow organ to be essentially expanded, the negative pressure having a level required to provide the suction and being specific for different hollow organs of mammals with different physiological features of such hollow organs, and allows prevention of partial or complete blockage or occlusion of the functional opening that may be caused by suction of the tissue of the hollow organ into the functional opening and that would result in necessity to retrieve the mounted catheter from the hollow organ of the mammal and, then, change the retrieved catheter to a new catheter or remove the hollow organ tissue entered into the functional opening from the functional opening of the retrieved catheter.

Furthermore, the treatment method according to any of the third, sixth and ninth aspects of the present invention provides a new technical effect which is improved utility of the treatment. The new technical effect is further provided by the treatment method according to any of the third, sixth and ninth aspects of the present invention due to the fact that the projections enclosing the functional opening positioned therebetween are provided between the balloons, and the enclosing projections are ring-shaped. In particular, the improved utility of the treatment is further provided by the treatment method according to any of the third, sixth and ninth aspects of the present invention due to the fact that the possibilities of production of the controlled negative pressure in the isolated interior of the hollow organ are essentially expanded, the negative pressure having a level required to provide the suction, and/or the fact that liquid or gaseous medium having a required dose or volume being specific for different hollow organs of mammals with different physiological features of such hollow organs and/or being specific for different deceases of hollow organs of mammals is supplied into the isolated interior, wherein such expanded possibilities are conditioned by increased volume of the isolated interior in the hollow organ of the mammal, the isolated interior being used for producing the controlled negative pressure therein and delivering the liquid or gaseous medium thereto.

Furthermore, the treatment method according to any of the third, sixth and ninth aspects of the present invention provides a further new technical effect which is the appropriate centering of the functional opening in relation to the target zone of the hollow organ of the mammal, the target zone being used for sucking the liquid or gaseous medium therefrom or used for delivering the liquid or gaseous medium thereto. In particular, the further new technical effect is further provided by the treatment method according to any of the third, sixth and ninth aspects of the present invention due to the fact that the projections enclosing the functional opening positioned therebetween are provided between the balloons, and the enclosing projections are ring-shaped.

### BRIEF DESCRIPTION OF THE DRAWINGS

While the specification concludes with claims particularly pointing out and distinctly claiming the present invention, it is believed the same will be better understood from the following description taken in conjunction with the accompanying drawings, which illustrate, in a non-limiting fashion, the best mode presently contemplated for carrying out the present invention, and in which like reference numerals designate like parts throughout the drawings, wherein:

FIG. 1 schematically illustrates a catheter for isolating a region in a hollow organ of a mammal according to the present invention;

FIG. 2 illustrates a functional opening provided in a part of the catheter of FIG. 1;

FIG. 3 illustrates the catheter with a grid enclosure in a state when balloons are deflated or blown off;

FIG. 4 illustrates the catheter with the grid enclosure in a state when balloons are inflated.

### DETAILED DESCRIPTION

In the context of this document, unless explicitly stated otherwise, the term “patient” means first of all a potentially sick person (a member of the mammalian class) seeking medical advice or remaining under medical observation to have a disease diagnosed and/or treated, wherein the term “patient” also means potentially sick mammalian animals remaining under medical observation to diagnose and/or treat their disease.

Furthermore, in the context of this document, unless expressly stated otherwise, the term “mammal” means a human or an animal, in particular anthropoid and non-human primates, dogs, cats, horses, camels, donkeys, cows, sheep, pigs, and other well-known mammals.

Furthermore, in the context of this document, unless expressly stated otherwise, the term “user” means any suitably skilled health care professional authorized to insert the catheter according to the present invention into a hollow organ of a mammal (in particular, a human hollow organ), remove the catheter according to the present invention from a hollow organ of a mammal and/or manipulate the catheter according to the present invention inserted into a hollow organ of a mammal, wherein the healthcare professional may be, for example, surgeon, oncologist, endoscopist, thoracic surgeon, angiosurgeon, urologist, veterinarian, etc.

Nowadays, the most advanced method of diagnosing and/or treating inflammatory, autoimmune, infectious, benign and/or malignant diseases occurring in hollow organs, or connected with them organs of a mammal, in particular diseases of pancreas, bile ducts, liver, gastrointestinal tract, and also defects and injuries of hollow organs of a mammal, fistulas or perforations, strictures, aneurismal and diverticular dilatations and/or etc., is a liquid biopsy based on the determination of disease-specific features in biological liquids or biological fluids of an organism being specific of a hollow organ or connected with it organs of a mammal. In particular, a liquid biopsy used to diagnose and/or treat pancreatic cancer of a mammal at an initial stage and precancerous transformation is based on the detection of circulating pathologic cells, circulating tumor DNA, RNA, proteins, peptides, metabolites, as well as circulating tumor exosomes in biological liquids of the organism of a mammal (such as blood and pancreatic juice).

One critical aspect of the liquid biopsy is the amount of sampled material, so that it is required to have a sufficient amount of the sampled material to perform the diagnosis. One more critical aspect of liquid biopsy is tumor localization since pathologic genetic or other diagnostic material isolated from a sample may be typical of cancers of different hollow organs. In particular, in view of the above reason, to diagnose a cancer, mucinous and intraepithelial neoplasms of the pancreas, pancreatic juice is the most appropriate diagnostic liquid to be used for detecting circulating tumor cells, DNA, RNA, proteins, peptides, metabolites, exosomes therein. However, sampling of pancreatic juice is the most difficult problem, which is effectively solved by the catheter 100 according to any one of the below-described embodiments of the present invention. Structural and design features and functionalities of the catheter 100 according to the present invention are described in details below in context of solving an illustrative task of collecting pancreatic juice from duodenal papillas, however the scope of the present invention is not limited by them.

It is to note that the minor duodenal papilla and the major duodenal papilla (also referred to in the art as Santorini's papilla and Fateri's papilla, respectively) each represent an anatomical structure in the form of a hemispherical, conical or flattened elevation located at the end of the longitudinal fold of the mucous membrane in the middle of the descending part of the duodenum, in particular about 12-14 cm below the pylorus, wherein in most cases one opening common for the bile and pancreatic ducts is exposed to the duodenum lumen, and in other cases the pancreatic duct is exposed 2-4 cm above the duodenal papilla. A hepato-pancreatic ampoule is located in the duodenal papilla, the ampoule being used for receiving bile and digestive juices of the pancreas and contains the Oddi sphincter regulating the flow of bile or pancreatic juice into the duodenum and preventing the intestinal contents from entering the bile and pancreatic ducts. Therefore, the major duodenal papilla in the duodenum of a mammal is usually 12-14 cm below the pylorus, and the minor duodenal papilla is 2-4 cm above the major duodenal papilla.

In particular, in order to provide the flow of pancreatic juice into the lumen of duodenum it is necessary to create physiological negative pressure around the Fateri's papilla or major duodenal papilla at a level of 40-100 mmH₂O, which is normally achieved by peristalsis of duodenum (Physiology of digestion. S. Teesalu. 1987. Tartu. Tartu State University. p. 84; The pancreas. Third edition. 2018. Blackwell. UK. 1300). Another criterion is the need for isolated sampling of a pancreatic juice with inactive (digestive) enzymes, without gastric and duodenal juice and contents, since otherwise presence of gastric and duodenal juice and contents will result in activation of enzymes and digestion of cells, DNA, aminoacids and exosomes necessary for diagnosis.

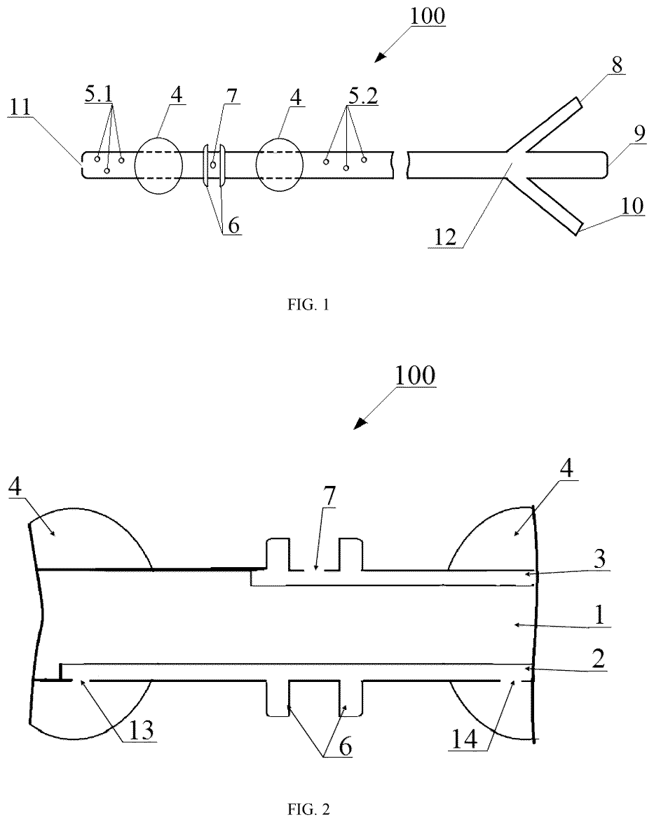

FIGS. 1-4 schematically illustrate a catheter 100 for isolating a region in a hollow organ according to the present invention, wherein the catheter 100 is a catheter to be inserted by a user into a lumen of a hollow organ, and wherein a housing or a body of the catheter 100 is formed as a flexible hollow tube having dimensions, in particular a length and a thickness, suitable for user-assisted insertion or advancement thereof within the lumen of a hollow organ towards a placement site. A user manipulating the catheter 100 may be an appropriately skilled healthcare professional, such as, for example, surgeon, oncologist, endoscopist, thoracic surgeon, angiosurgeon, urologist, veterinarian, etc. In particular, the catheter 100 for isolating the region in the hollow organ may be used for any patient, in particular any human or animal related to a mammal.

The catheter 100 of FIG. 1 is provided at its distal end with an axial opening 11, the distal end being used for administering or inserting the catheter 100 into a patient followed by advancing the catheter 100 to a placement site within the lumen in a patient's hollow organ, in particular in the gastrointestinal tract, bile ducts, respiratory tracts, urinary system, patient vessels, a cavity related to uterine and vagina, etc. Furthermore, the catheter 100 is fitted with a three-way connector 12 at its proximal end opposite to the distal end of the catheter 100, wherein the proximal end is located outside of patient's body when the catheter 100 is inserted into the lumen of patient's hollow organ.

As shown in FIG. 1, the three-way connector 12 in the catheter 100 may be a pipe or a tube provided with three branches or terminals: a central terminal 9, and two side terminals 8 and 10 hermetically isolated from the central terminal 9, wherein terminals 8, 9, 10 are each provided with a corresponding opening at their free end and each configured to connect to or join an appropriate functional appliance or device thereto.

The central terminal 9 positioned between the side terminals 8, 10 in the three-way connector 12 is configured to connect to or join an appropriate (first) functional appliance or device thereto, wherein the first functional device may be any device known in the art for supplying a gaseous medium or fluid (not shown), for example an enteral nutrition mixture. The device for supplying a gaseous medium or fluid as connected to the central terminal 9 may be, for example, an injection syringe filled, for example, with an enteral nutrition mixture to provide patient's organism with basic nutrients, energy, vitamins, macronutrients, micronutrients and/or etc., or a medical dropper filled, for example, with an enteral nutrition mixture, or other devices and appliances, including automated or semi-automated, suitable to connect the central terminal 9 thereto and supply a gaseous medium or fluid, for example an enteral nutrition mixture, to the central terminal 9. Therefore, the device for supplying a gaseous medium or fluid connected or attached to the central terminal 9 may be used to provide, for example, nutrition support or clinical nutrition to a patient when the catheter 100 is inserted within the lumen of duodenum (or other part of gastrointestinal tract). Furthermore, the device for supplying a liquid or gaseous medium, when connected or attached to the central terminal 9, may be used, for example, to sanitize the stomach and patient's duodenum.

The side terminal 8 being one of the two side terminals in the three-way connector 12 is designed to connect or attach to an appropriate (second) functional appliance or device thereto, wherein the second functional device may be implemented, for example, as a medical suction apparatus, an aspiration device or an aspirator (not shown) comprising of a storage reservoir or a storage container (not shown) for collecting biological gaseous medium, biological liquid and/or biological fluid and an air compressor (not shown) for aspirating or evacuating an air or another appropriate gaseous medium. The pressure for aspirating or evacuating the air or another appropriate gaseous medium may be adjusted by a one skilled in the art for corresponding hollow organs on the basis of information disclosed in the prior art documents, for example, in U.S. Pat. No. 6,712,798.

Furthermore, the second functional device to be attached to the side terminal 8 in the three-way connector 12 may be formed as a special device or device for supplying a gaseous medium or fluid (for example, medical products), for example an injection syringe filled with a liquid to be supplied, or a medical dropper filled with a liquid to be supplied, or other devices or appliances, including automated or semi-automated, suitable for connecting the side terminal 8 thereto and supplying said gaseous medium or fluid to the side terminal 8.

The other side terminal 10 in the three-way connector 12 is configured to connect or attach to an appropriate (third) functional appliance or device thereto, wherein the third functional device may be implemented as a special device or a device for supplying a liquid or gaseous medium under pressure (not shown), for example water or air, in particular an injection syringe filled with a liquid or gaseous medium (for example, water or air) to be supplied, or a medical dropper filled with a liquid (for example, water) to be supplied, or other devices and appliances, including automated or semi-automated, suitable for connecting the side terminal 10 thereto and supplying a fluid or gaseous medium to the side terminal 10.

It is to note that the above-described first functional device (not shown) connectable to the central terminal 9 of the catheter 100 for supplying a liquid, for example a enteral nutrition mixture, and the above-described second functional device (not shown) connectable to the side terminal 8 of the catheter 100 for sampling a biological fluid or biological gaseous medium or supplying a liquid or gaseous medium, and/or the third functional device (not shown) connectable to the side terminal 10 of the catheter 100 for supplying a liquid, for example water, in combination with the catheter 100 shown in FIGS. 1-4 may form a system for isolating a region in a hollow organ of a mammal (not shown), which may be used for sampling a biological fluid or biological gaseous medium (for example, a biological liquid) being specific of certain hollow organ of a mammal, or may constitute corresponding parts of such a system. In particular, the above system may be used for isolating a region in the duodenum of a mammal for sampling pancreatic juice and/or bile.

Furthermore, as shown in FIGS. 1-4, the body of the catheter 100 is provided at its external side with two isolating balloons 4, each formed as an expanding or inflating soft reservoir, wherein isolating balloons 4 are spaced at a predetermined distance from each other and from the distal end of the catheter 100. A functional orifice or opening 7 is provided between the isolating balloons 4 in the body of the catheter 100. When the catheter 100 is inserted into a lumen of a patient's hollow organ, one of the isolating balloons 4, namely the farthest from the distal end of the catheter 100, becomes positioned farther than the region of interest in the hollow organ, and the other isolating balloon 4 becomes isolated up to the region of interest in the hollow organ, wherein the functional opening 7 is opposite to the region of interest or next thereto, for example opposite or adjacent to one of the minor and major duodenal papillas, between the minor duodenal papilla and the major duodenal papilla, opposite or adjacent to an intestinal fistula, opposite or adjacent to a wall lesion, opposite or adjacent to a tumor, etc.

Furthermore, the catheter 100 for isolating a region in a hollow organ comprises of three separate functional channels provided in the elongate body of the catheter 100: a main channel 1, a supply channel 2 for supplying a liquid or gaseous medium to the isolating balloons 4, the supply channel 2 being hermetically isolated from the main channel 1 and having holes provided in the catheter body 100 and each opening into the interior of one of the corresponding isolating balloons 4, and a functional channel 3 separated from the supply channel 2 and hermetically isolated from the main channel 1. The supply channel 2 and the functional channel 3 extend within a part of the main channel 1 along its length.

The main channel 1 extending substantially along all length of the catheter 100 communicates with the central terminal 9 of the three-way connector 12, wherein the main channel 1 has an inlet hole or an inlet corresponding to an opening provided in the central terminal 9 at the proximal end of the catheter 100 and has an outlet hole or an outlet corresponding to the axial hole 11 at the distal end of the catheter 100. When the catheter 100 is inserted into the lumen of patient's hollow organ, the inlet of the main channel 1 is located outside of the patient body to communicate with an ambient atmosphere or environment, and the outlet of the main channel 1 communicates with the lumen of the organ, for example esophagus, stomach and duodenum, small intestine or large intestine, and also respiratory tracts, biliary tract, urinary tracts, vessels, etc. Therefore, in case when the above-described first functional device (not shown) is connected to the central terminal 9 of the three-way connector 12, medical products, in particular enteral or parenteral nutrition mixtures, may be initially supplied under pressure from the first functional device to the central terminal 9 through the hole made therein, then supplied from the central terminal 9 to the main channel 1, and finally supplied from the main channel 1 to the interior of the hollow organ through the axial hole 11 shown in FIG. 1.

The supply channel 2 extending substantially along a part of the length of the catheter 100 communicates with each of the isolating balloons 4 through one of the corresponding outlets 13, 14 provided in the body of the catheter 100 (as shown in FIG. 2) and communicates with the side terminal 10 of the three-way connector 12, wherein the supply channel 2 has an inlet hole or an inlet corresponding to an opening provided in the side terminal 10 at the proximal end of the catheter 100. When the catheter 100 is inserted into the lumen of patient's hollow organ, the inlet of the supply channel 2 is located outside of the patient body to communicate with an ambient atmosphere or environment. Therefore, in case when the above-described second functional device (not shown) is connected to the side terminal 10 of the three-way connector 12, a gaseous medium or fluid, for example a air or water, in a given volume may be firstly supplied under pressure from the second functional device to the side terminal 10 through the hole made therein, then supplied from the side terminal 10 to the supply channel 2, and finally supplied from the supply channel 2 to both isolating balloons 4 through the corresponding holes made in the catheter body 100, so that the isolating balloons 4 are inflated or filled with water or a gas by using, in particular, the supply channel 2 of the catheter 100 to provide collectively filled or inflated balloons 4 each having an increased size or volume. The pressure used for filling or inflating the isolating balloons 4 with a fluid or gas for any particular hollow organ may be selected by one skilled in the art on the basis of information disclosed in prior art documents, for example, in U.S. Pat. No. 7,722,568.

It is to note that an increase in size or volume of the isolating balloons 4 can result in bilateral obturation or blockage of the lumen of patient's hollow organ, for example the lumen of patient's duodenum where the catheter 100 is inserted, thereby allowing a part of the catheter 100 with a functional opening 7 to be isolated between the inflated isolating balloons 4 in the patient's hollow organ. Therefore, the inflated isolating balloons 4 allow a section of patient's hollow organ, for example major and minor duodenal papillas of the duodenum, aneurysm, hollow organ's wall lesion, etc., to be isolated from proximal and distal sections of the hollow organ and, therefore, they exclude or prevent the targeted biological liquid from mixing with other biological liquids and allow the catheter 100 to be fixed within the lumen of the patient's hollow organ, for example within the lumen of patient's duodenum, due to close adjoining of the balloons 4 by their external sides to an inner wall surface of a hollow organ. In particular, in case when the catheter 100 is inserted into the lumen of duodenum, the inflated isolating balloons 4 allow the minor duodenal papilla and/or the major duodenal papilla of patient's duodenum to be isolated from proximal and distal sections of the gastrointestinal tract and, therefore prevent mixing of the target biological liquid, wherein the target biological liquid may be a mixture of pancreatic juice and bile, with other biological liquids, such as gastric juice and contents and/or duodenal contents. This ensures that digestive enzymes contained in pancreatic juice remain inactive.

In one embodiment of the present invention, the isolating balloons 4 may communicate therebetween through the supply channel 2, thereby ensuring uniform redistribution of a liquid or gaseous medium used to expand or inflate them, for example, during the passage of a peristaltic wave of the intestine or esophagus. In another embodiment of the present invention, the isolating balloons 4 may be formed, for example, as self-inflating balloons or balloons inflated by air supplied to the supply channel 2 of the catheter 100 through the hole in the side terminal 10 of the three-way connector 12, or a balloon inflated in any other way known in the art.

In one of the embodiments of the present invention, the side terminal 10 in the three-way connector 12 may be optionally equipped with a manually operated shut-off valve (not shown) to prevent the backflow or escape of a gaseous medium or fluid, in particular a gas or water located in the isolating balloons 4, the supply channel 2 and the side terminal 10. It is to further note that the isolation valve may be used by a catheter user to adjust an inflation degree of the isolating balloons 4, wherein the user may visually control the inflation degree by using an endoscope. In particular the user may reduce sizes or volumes of the inflated isolating balloons 4 by deflecting or venting a gaseous medium or fluid, in particular a gas or water, from the supply channel 4 by manually opening the check valve.

The functional channel 3 extending substantially along a part of the length of the catheter 100 communicates with the side terminal 8 of the three-way connector 12, wherein the functional channel 3 has an inlet or an inlet port corresponding to the functional opening 7, and an outlet or an outlet port corresponding to an opening provided in the side terminal 8 at the proximal end of the catheter 100. Therefore, in case when the above-described third functional device (not shown) implemented as an aspirator is connected to the side terminal 8 of the three-way connector 12, the functional channel 3 will substantially serve as an aspiration channel, and the functional opening will substantially serve as an aspiration opening, wherein a negative air pressure or a negative air medium pressure will be produced or supported in the interior of the hollow organ, the hollow organ interior being isolated by the inflated isolating balloons 4, as described above. The negative pressure (also referred to in the art as a technical vacuum) produced in the isolated interior of patient's hollow organ promotes the evacuation or removal of biological gaseous medium or biological fluid, in particular biological liquid, such as bile and pancreatic juice or pancreatic secretion or pus, or blood, or bronchial secretion, to firstly evacuate said gaseous medium or fluid from the isolated interior to the functional channel 3 through the functional opening 7, then from a functional channel 3 to the side terminal 8, and finally from the side terminal 8 to a storage container of the aspirator through the corresponding hole made in the side terminal 8. It is to note that the functional channel 3 with a functional opening 7 may be used not only for sampling a biological gaseous medium or biological fluid being specific of a particular hollow organ, but also for supplying a required fluid or gaseous medium, for example a drug in a liquid or gaseous form or coloring agent, to the isolated interior in case when the above-described third functional device (not shown) is implemented as a device for supplying a gaseous medium or fluid, for example a medical syringe or medical dropper, is connected to the side terminal 8 of the three-way connector 12.

It is to note that the aspirator being one of possible implementations of the above-described third functional device connectable to the side terminal 8 of the three-way connector 12, when activated by the user, activates an air compressor being a part of the aspirator. The activated air compressor provides aspiration or evacuation of air medium or air from the interior of patient's hollow organ, for example from the interior of patient's duodenum isolated by the inflated isolating balloons 4 in the above-described manner, and from an available space in the aspirator storage container, the available space being not filled with a biological gaseous medium or biological fluid (for example, a biological fluid, such as a bile and a pancreatic juice, or a pancreatic secretion) to produce a negative pressure in the isolated interior of a hollow organ, for example in the lumen of patient's duodenum, thereby taking the biological medium through the functional opening 7 and removing or supplying the taken biological medium, by the functional channel 3, to the storage container for accumulating or collecting therein for a predetermined time period.

It is to note that in one of the embodiments the side terminal 8 of the three-way connector 12 may be used for administering therethrough a liquid or gaseous substance carried by the functional channel 3 and entering the interior between the isolating balloons 4 through the functional opening 7. The administered gaseous or liquid substance, for example a drug or coloring agent, may affect the region of a hollow organ isolated by the balloons 4 and have a diagnostic and/or therapeutic effect thereon.

In one of the embodiments of the present invention, only the above-mentioned supply channel 2 for supplying a fluid (for example, water) to the isolating balloons 4 communicating with the above-described side terminal 10 and the above-described functional channel 3 communicating with the above-described side terminal 8 may be provided in the catheter body 100.

Furthermore, the body of the catheter 100 is provided at its external side with two generally identical bulges 6, each formed as a ring-shaped projection or a ring having a radius which is more than that of the body of the catheter 100 or less than that of any one of the inflated isolating balloons 4, wherein each of bulges 6 is located along a length of the body of the catheter 100 on one side in relation to the functional opening 7 next or close thereto, and wherein the bulges 6 are preferably equally spaced in relation to the functional opening 7 between the isolating balloons 4. It is to note that in case when the catheter 100 is inserted into the lumen of patient's hollow organ, bulges 6 serving substantially as projecting sides enclosing the functional opening 7 prevent the functional opening 7 from being contacted with or from approaching the mucosa of patient's hollow organ for a distance appropriate for suction of the mucosal tissue into the functional opening 7 when an aspirator connected to the functional channel 3 of the catheter 100 is activated by the user and, therefore, when the negative pressure is provided by an aspirator within the isolated interior in the lumen of a hollow organ that would damage mucosal tissue of a hollow organ. In one embodiment of the present invention, the bulges 6 may have any other form different from a ring or ring-shaped form provided that such a form prevents the functional opening 7 from being contacted with the mucosa of patient's hollow organ or prevents the functional opening 7 from approaching the mucosa of patient's hollow organ for a distance appropriate for suction of the mucosa into the functional opening 7 when producing a negative pressure in the isolated interior of a hollow organ communicating with the functional opening 7.

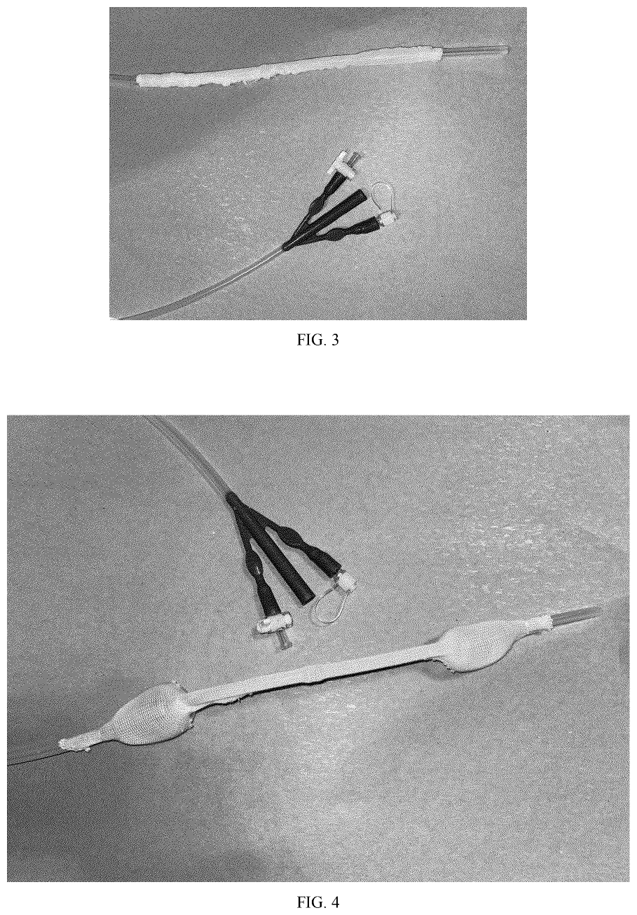

Furthermore, the body of the catheter 100, as shown in FIGS. 3-4, is provided at its external side with two protrusions or projections 15, wherein each of the projections 15 is provided outside a catheter body part defined by the isolating balloons 4 at a predetermined distance from one of the corresponding balloons 4, wherein an elastic net or a grid enclosure 16 is attached to the projections 15 to completely or at least partly cover both the isolating balloons 4 and a part of the body of the catheter 100 defined by the balloons 4, thereby completely or at least partly enveloping or covering the functional opening 7. When the balloons 4 are inflated, as shown in FIG. 4, the grid enclosure 16 becomes strained or stretched, thereby moving or displacing the inner wall tissue in a hollow organ for a predetermined distance away from the functional opening 7. The grid enclosure 16 in a completely or at least partly strained state forms an elastic outer casing or frame which completely or at least partly encloses the catheter body part defined by the balloons 4 or located between the balloons 4, thereby completely or at least partly enveloping or covering the functional opening 7. The outer frame formed by the grid enclosure 16 has a cylindrical shape and is penetrable by a liquid due to its cellular structure, thereby allowing the biological fluid specific of a certain hollow organ of a mammal to penetrate or pass through a material of the grid enclosure 16 and to enter the functional opening 7. In particular, in case when an aspirating device (not shown) is connected to the side terminal 8 to provide, by the functional channel 3 and the functional opening 7 in the catheter 100, a negative pressure in the isolated region between the isolating balloons 4, the stretched or strained grid enclosure 16 will hinder glueing or adhesion of the inner wall tissue in a hollow organ (for example, vessel walls, bowel mucosa, bronchus, stomach, ureter, or etc.) to the functional opening 7, thereby allowing the constant aspiration of biological material or biological fluid, for example, bile, pancreatic juice, bronchial secretions, etc., into the container of the aspirating device.

In one embodiment of the present invention, the grid enclosure 16 may be attached to both isolating balloons 4 such that it completely or at least partly encloses a catheter body part defined by the balloons 4 or located between the balloons 4, thereby completely or at least partly enveloping or covering the functional opening 7. When the balloons 4 are inflated, as shown in FIG. 4, the grid enclosure 16 becomes strained or stretched, thereby allowing for removal or offsetting of the inner wall tissue in a hollow organ for a predetermined distance away from the functional opening 7.

In another embodiment of the present invention, the grid enclosure 16 may be formed as a net-like material or a net preliminary secured in a strained state or at least partly strained state on the bulges 6 such that it completely or at least partly encloses a catheter body part defined by the balloons 4 or located between the balloons 4, thereby completely or at least partly enveloping or covering the functional opening 7.

In some other embodiments of the present invention, the grid enclosure 16 may be secured, any suitable fastening means known in the art, in a preliminary strained state on the body of the catheter 100 such that it completely or at least partly encloses a catheter body part defined by the balloons 4 or located between the balloons 4, thereby completely or at least partly enveloping or covering the functional opening 7.

In other embodiments of the present invention, the grid enclosure 16 may have any form allowing the grid enclosure 16 to be secured on the body of the catheter 100, on the bulges 6 or on the isolating balloons 4 to completely or at least partly envelop or cover the functional opening 7.

Furthermore, three auxiliary holes 5.1, 5.2 are provided in the catheter body 100 outside of the isolating balloons 4 and, therefore, outside of the catheter body part provided with the functional opening 7 and defined by the isolating balloons 4. When the catheter 100 is inserted into the lumen of patient's hollow organ, the auxiliary holes 5.2 located further from the distal end of the catheter 100 serve as inlets, and the auxiliary holes 5.1 positioned closer to the distal end of the catheter 100, serve as outlets. It is to note that auxiliary hole 5.1, 5.2 allow a hollow organ contents for example air, urine, blood, gastric or intestinal contents with inactive digestive ferments to pass to distal sections of a hollow organ without entering the region defined by the inflated isolating balloons 4 of the catheter 100, thereby preventing or eliminating the formation of congestive and/or inflammatory processes in patient's hollow organ that are caused, in particular, by accumulation of blood, urine, air, mucus and other contents in patient's hollow organ outside of the inflated isolating balloon 4 located farther or farthest from the distal end of the catheter 100.

Depending on the use of the catheter 100 according to the present invention and on anatomical parameters of the hollow organ, a length and diameter of the catheter 100, a wall thickness of the catheter 100, a location and diameter of the isolating balloons 4 in the catheter 100, and the location of the holes may vary. One skilled in the art may easily adjust any parameter of the catheter 100 depending on the dimensions of a particular hollow organ.

The thickness of the channels of the catheter 100 and the size of the isolating balloons 4 may be adjusted by one skilled in the art on the basis of information disclosed in the art, for example, U.S. Pat. Nos. 9,526,874, 6,692,465, 5,843,050, 5,919,163, international publication WO 2009/035581, U.S. Pat. Nos. 5,397,305, 8,398,589, 7,722,568, 6,712,798, 6,638,245, 1,009,865 and/or other prior art information sources.

The length of the catheter 100, a distance between the isolating balloons 4, and a distance from the isolating balloons 4 to the distal end may be matched by one skilled in the art on the basis of information disclosed in the art, for example, U.S. Pat. Nos. 5,314,409, 5,658,264, US patent application No. 20150150572, U.S. Pat. Nos. 5,843,050, 5,397,305, 7,070,606, 6,712,798, 1,009,865 and/or other prior art information sources.

For example, in one of the preferable embodiments of the present invention, the catheter 100 may be implemented as a pancreato-digestive catheter and may have a length of 130 cm. In the present embodiment of the present invention, the isolating balloons 4 are correspondingly spaced at 15 sm and 25 sm from the distal end of the catheter 100, the distal end being used for inserting the catheter 100 into the gastric cavity and the lumen of patient's duodenum, so that expanding or inflating of the isolating balloons 4 allows for isolation of a duodenum region having a length of at least 10 cm. In the present embodiment of the present invention, the functional opening 7 may be in the middle between the isolating balloons 4, i.e. at a distance of 5 cm from each of the isolating balloons 4 and at a distance of 20 cm from the distal end of the catheter 100.

Use of the catheter 100 according to the present invention for isolating a region in patient's hollow organ is illustratively described above in relation to patient's duodenum. However the use of the catheter 100 is not limited by patient's duodenum. Therefore, it is clear for one skilled in the art that the catheter 100 according to any of the above-described embodiments can be similarly inserted into a lumen of any another hollow (tubular) internal organ of a patient, in particular mammals, for example in the esophagus, stomach, duodenum, small intestine, large intestine, respiratory tracts, urinary tracts (urogenital system tracts), veins, arteries, vagina, uterus, uterine (Fallopian) tubes, vertebral canal or any appropriate internal tubular organ of a patient, the patient tubular organ being related to a corresponding functional system (apparatus of organs) of a mammal organism from a group of systems including: digestive system, respiratory system, urinary and reproductive systems (combined into the genitourinary system or urogenital system), endocrine system, circulatory system and immune system, and skeletal system.

Therefore, when used, the catheter 100 according to the present invention may be inserted by the user into the lumen of a patient's duodenum under control of an endoscope (not shown) or of a radiographic equipment (for example, a fluoroscopy equipment) such that one of the corresponding isolating balloons 4, the farthest from the distal end of the catheter 100, is located in the bulb of patient's duodenum, wherein the endoscope may be manipulated by the user or endoscopist assisting the user. In particular, it is to note that the insertion process of the catheter 100 into the lumen of patient's duodenum, the removal process of the catheter 100 from the lumen of patient's duodenum after sampling a required amount the biological liquid and the aspiration process are atraumatic and do not depend on the anatomical features of both the patient and neoplasms. It is to note that the isolating balloons 4 are in deflated state when the catheter 100 is inserted by the user into the lumen of patient's duodenum.

According to one example, the catheter 100 may be preliminary equipped with at least one loop designed to grip it with biopsy forceps. To insert the catheter 100 into the desired placement site in patient's duodenum the distal end of the catheter 100, well lubricated with vaseline oil, is administered through the nasal passage and advanced to the gastric cavity; then the endoscope is further administered or advanced in parallel with the catheter 100 through the patient oral cavity to the patient's gastric cavity to capture, by means of the endoscope biopsy forceps, the loop(s) of the catheter 100; finally the captured catheter 100 is guided or advanced along with the endoscope to patient's duodenum. Subsequently, under control of an endoscope, one of the corresponding isolating balloons 4 of the catheter 100, the farthest from the distal end of the catheter 100, is placed in the bulb of patient's duodenum.

According to another example, a metal guidewire may be preliminary administered or inserted into the main channel 1 of the catheter 100. To insert the catheter 100 into a desired placement site in patient's duodenum the distal end of the catheter 100, well lubricated with vaseline oil, is inserted through the nasal passage and then advanced to the gastric cavity by using the metal guidewire of the catheter 100; then the endoscope is inserted or advanced in parallel with the catheter 100 through the patient oral cavity to the patient gastric cavity to capture, by means of the endoscope biopsy forceps, a first ligature upon detection of the distal end of the catheter 100 and to press the catheter 100 to the endoscope by pulling up the catheter 100 by using the captured first ligature. Subsequently, the endoscope and the catheter 100 pressed against the endoscope are guided through the pylorus to the patient's duodenum, and one of the corresponding isolating balloons 4 of the catheter 100, the farthest from the distal end of the catheter 100, is placed under control of the endoscope within the bulb of patient's duodenum.

Then, the user connects or attaches the above-described second functional device (not shown) to the side terminal 10 of the three-way connector 12, wherein the second functional device is intended to supply a liquid or gaseous medium, for example a gas or water, to the supply channel 2 of the catheter 100 in a required amount or volume, in particular water in a volume of 40-70 ml, to allow the expansion and inflation of the isolating balloons 4 to closely adjoin to a mucosa of the duodenum, thereby isolating required region of patient's duodenum containing the major duodenal papilla and the minor duodenal papilla, in particular preventing gastric contents and/or duodenal contents with inactive digestive ferments from entering the isolated region. In particular, it is to note that results experimentally obtained by the inventors show that 40-70 ml of air is required to sufficiently inflate the isolating balloons 4 to a required size for bilateral obturation or occlusion of patient's duodenum lumen where the catheter 100 may be inserted, as an illustrative example. Then, the user joins or connects the above-described third functional device (not shown) implemented as an aspirator to the functional channel 3, wherein aspirator is preliminary configured for a constant or variable operation mode providing a required working pressure depending on a particular task. The aspirator activated by the user allows an air medium or air to be sucked, through the functional opening 7 and the functional channel 3 communicating therewith, from the isolated interior of the patient duodenum, the isolated interior corresponding to the periampullary zone of the duodenum, thereby producing in the interior negative pressure corresponding to pressure normally created by the peristalsis of the duodenum, in particular a negative pressure of 40-100 mmH2O (9-14 kPa)). The negative pressure provided in the isolated interior of patient's duodenum allows a biological liquid in the form of pancreatic juice or pancreas secretion in combination with bile to be evacuated or retrieved from the pancreas through the major duodenal papilla and the minor duodenal papilla, thereby removing said biological liquid, through the functional opening 7 and the functional channel 3, from the isolated region of patient's duodenum and collecting it in a storage container (not shown) of the aspirator. In some cases, there may be further collected in the container a mucosal secretion of the bile and pancreatic ducts, the mucosal secretion being one of the varieties of the biological liquid being specific of duodenum, and/or some biological contents which would enter the bile and the pancreatic ducts as a result of reflux and would contain liquids produced by the organism, the liquids being specific of human duodenum. Subsequently, the biological liquid collected in the storage container of the functional device (not shown), can be delivered for cytological examination and/or molecular genetic examination or other analyses to evaluate characteristics of a morphological structure of cellular elements of pancreatic secretion, cell elements allowing one to detect or reveal pathologic processes being specific, for example, of intraductal neoplasms, neuroendocrine tumors or pancreatic cancer, and to detect neoplasms at an early stage of development, and to perform differential diagnosis between different types of tumors by determining expressions of specific markers (NKX2, S100P, CEA, EFR3A/B, MUC1, MUC2, MUC5, ANXA1, A2, KRT7, MMP7, MMP9, IGFBP3, PSCA, PRSS2, SHh, KRas, TP53, SMAD4, BRCA1, miRNA 21, and miRNA 155).

It is to note that the catheter 100 according to the present invention may remain in the inserted state for a sufficiently long period of time, for example up to seven days (i.e. up to 168 hours), allowing for the pancreatic secretion to be collected in an appropriate amount for reliable identification and verification of pathologic tumor material, so that it generally improves the efficiency of diagnosis and, therefore, subsequent treatment of pancreatic cancer. Furthermore, prolonged sampling increases the likelihood of detecting pathologic cells and other biomarkers not only for pancreatic cancers, but also for malignat conditions of bile ducts and liver, including cholangiocarcinomas and/or hepatocellular cancer.

It is to further note that stimulation of the secretion of pancreatic juice and bile is not required when the catheter 100 is used, so that a biological material to be collected has a true biochemical and physiological profile presenting a true functional state of pancreas, liver, bile ducts, contractility of the gallbladder.

If necessary, the excretory function of pancreas may be evaluated by examining a biochemical composition of pancreatic secret collected in the above-described manner in the storage container (not shown) in combination with the bile by using the catheter 100 according to the present invention.

Furthermore, a qualitative and quantitative analysis of the pancreatic juice and bile allows the true biochemical profile of both biological materials and a volume of daily secretion to be evaluated, thereby diagnosing a functional condition of an organ.

In particular, when the catheter 100 is used, there is provided a treatment or therapeutic effect achieved by restoring and facilitating the evacuation of bile or pancreatic juice from corresponding ducts into duodenum. The pathogenesis of acute inflammatory diseases, such as pancreatitis and cholangitis, is caused at least in part by the impairment or disruption of the outflow of pancreatic juice and bile, the outflow impairment being caused, in particular, by impaired motility (peristalsis) of duodenum of a mammal, so that the isolated region having a controllable negative pressure (for example, 40-100 mmH₂O) produced when performing aspiration of the pancreatic juice and abile by using the catheter 100 allows this medical problem to be solved and, therefore, to contributes to the treatment of the above acute diseases.

Furthermore, when the catheter 100 is used, there is provided a treatment or therapeutic effect achieved by evacuating contents from a region pertaining to a wall lesion of gastrointestinal tract, so that the isolated region having a controllable negative pressure (for example, at a level of 70-100 mm water column) allows all the biological liquids to be evacuated from a lesion region and allows this medical problem to be solved, thereby contributing to solving said medical problem of a patient.

Therefore, the above-described catheter 100 allows a functional investigation of any hollow organ to be conducted or performed and allows appropriate treatments to be provided. Furthermore, examination and/or laboratory analysis of a biological liquid collected by using the catheter 100 allows inflammatory formations, benign formations, malignant growth and infectious diseases to be differentially diagnosed with a higher accuracy. Due to evacuation of a specific liquid, the catheter 100 allows the treatment of inflammatory diseases, bleeding and lesions of hollow organ walls. Producing an isolated region having a function of bypass during a surgical manipulation allows hemostasis to be controlled and allows surgical manipulations, in particular resection of a site of vessels and subsequent plasty of the vessels, to be performed. It is to further note that the catheter 100 according to the present invention or the above-described system for isolating a region in a hollow organ of a mammal (not shown), the system including the catheter 100, allows a high volume of a biological material in a normal physiological and biochemical state to be collected without any additional stimulation of the excretory function of the hollow organ, in particular due to the creation of a negative pressure in the isolated region of the hollow organ having the catheter 100 inserted into the lumen of the hollow organ, wherein the created negative pressure corresponds to the physiological negative pressure which may be produced, for example, due to the peristalsis of intestine.

### Uses of the Catheter for Treatment of Bleeding Into an Esophageal or Intestinal Lumen

It is to be noted that the catheter 100 according to any one of the above-described embodiments can be used for treatment of bleeding into an esophageal or intestinal lumen. In particular, the catheter 100 according to any one of the above-described embodiments can be further used for treatment of bleeding into a lumen of any of the bowel segments depending on a location of a source of bleeding in the intestine, including treatment of bleeding into a small intestine lumen or treatment of bleeding into a large intestine lumen. Furthermore, the catheter 100 according to any one of the above-described embodiments can be used for treatment of bleeding into a lumen of any of small intestine subdivisions, including bleeding into a duodenum lumen, bleeding into a jejunum lumen, and bleeding into a ileum lumen, and can be used for treatment of bleeding into a lumen of any of large intestine subdivisions, including bleeding into a cecum lumen, bleeding into a colon lumen, bleeding into a ascending colon lumen, bleeding into a transverse colon lumen, bleeding into a descending colon lumen, bleeding into a sigmoid lumen, and bleeding into a rectum lumen.

In particular, a method for treatment of bleeding into the esophageal or intestinal lumen based on the use of the above-described catheter 100 is a treatment technique based on a combination of mechanical action and topical pharmacological use of hemostatic agents. In particular, the inflation of the isolating balloons 4 provides a partial compression of the submucosal vasculature without traction of the catheter 100, so that the critical compression is eliminated. Due to the formation of the isolated region between the isolating balloons 4, precise visualization of the source of hemorrhage is not required (i.e., in general, it does not matter whether the source of hemorrhage is an arterial vessel, a venous vessel, a tumor site, a mucous membrane site, or the like), wherein the blood flowing into said isolated region prevents clots from being migrated and ensures the development of hemostasis both mechanically and through the action of coagulation factors. In addition, a hemostatic agent (e.g., the Hemocer hemostatic agent, the Arista hemostatic agent, the Hemoclot hemostatic agent, or the like) can be introduced into the isolated region formed between the isolating balloons 4, wherein the hemostatic agent forms a protective scab. Meanwhile, due to the inflated isolating balloons 4, the risk of migration of the hemostatic agent is eliminated, thus reducing the risk of repeated bleeding. The perforations provided in the main channel 1 outside the isolating cylinders 4 allow the draining of portions of the gastrointestinal tract, the gastrointestinal tract portions adjoining the isolated region from the outside, and further allow the effectiveness of hemostasis to be monitored and an enteral feeding to be performed.

### Uses of the Catheter for Treatment of an Esophageal or Intestinal Wall Fistula/Perforation

It is to be further noted that the catheter 100 according to any one of the above-described embodiments of the present invention can be used for treatment of an esophageal wall perforation or fistula or for treatment of an intestinal wall perforation or fistula, wherein the fistula is also referred to in the prior art as a sinus or lesion. In particular, the catheter 100 according to any one of the above-described embodiments of the present invention can further be used for treatment of the fistula in any of bowel segments depending on a location of the fistula in the intestine, including treatment of the fistula in the small intestine or treatment of the fistula in the large intestine. In addition, the catheter 100 according to any one of the above-described embodiments can be used for treatment of the fistula in any of subdivisions of the small intestine, including duodenal fistula, jejunal fistula, and ileal fistula, and can be also used for treatment of the fistula in any of subdivisions of the large intestine, including cecal fistula, colon fistula, ascending colon fistula, transverse colon fistula, descending colon fistula, sigmoid fistula, and rectal fistula.

In particular, the method for treatment of an esophageal or intestinal wall fistula/perforation based on the use of the above-described catheter 100 is a treatment technique based on the following main operations:
(1). Inflating the isolating balloons 4 so as to form an isolated region therebetween, the isolated region surrounding or containing the fistula or perforation of the intestinal or esophageal wall, thus preventing the ingress of content (e.g., saliva, gastric juice, bile, pancreatic juice, chyme, and/or the like) to said wall lesion, which in turn ensures the sparing of the wound caused by said wall lesion and provides the most optimal conditions for its healing. Forming the isolated region between the isolating balloons 4 prevents a biological media from entering said isolated region of the hollow organ, thereby reducing the risk of contamination of the wall lesion by a flora from other parts of the hollow organ outside said isolated region. Furthermore, the absence of content in the isolated region between the isolating balloons 4 leads to the esophageal, gastric and/or intestinal content ceasing to flow outside the examined organ, so that inflammation and peritonitis in other cavities and areas of the body are no longer maintained. In addition, the loss of proteins, fats, carbohydrates and microelements is stopped, so that additional development of a complex technique for reinfusion or replacement thereof is not required.
(2). The functional opening 7 allows a reduced pressure in the isolated region surrounding the esophageal or intestinal wall lesion to be formed, wherein the functional opening 7 is exposed to the isolated region so as to allow the communication between the functional channel 3 and the isolated region when the catheter 100 is installed. Similar to the EVAC therapy, the reduced pressure created around the esophageal or intestinal wall lesion within the isolated region allows the reverse drainage into the cavity through the organ surrounding cavity lesion and removes a mucus secreted by epithelial goblet cells. In addition, the functional opening 7 allows the lesion of the wall to be topically affected with various drugs, e.g., antiseptics, wherein isolating the region containing said organ wall lesion increases the time of exposure of the drug to the organ walls and the lesion itself. Furthermore, the functional opening 7 further allows the local administration of contrast media, which may be necessary both to control the placement of the catheter 100 in the hollow organ and to control the dynamics of the healing process of the wound caused by a fistula or a lesion in the wall of the esophagus or intestine.
(3). The drainage of the hollow organ sections adjacent to the isolated region is provided such that it ensures their communication with each other, preventing the development of mechanical obstruction of the esophagus, stomach, intestine or the like in the patient depending on a specific location of the gastrointestinal tract lesion. The communication between the sections of the hollow organ located outside the isolated region formed between the balloons 4 is necessary both for independent flow of biological fluids between said outside sections of the hollow organ and for enteral feeding of the patient. The enteral feeding of the patient is an extremely important aspect of treatment since it facilitates the correction in the composition of the received food and ensures that nutrition is more complete, thereby reducing the cost of the entire treatment process for the patient since parenteral nutritional mixtures are more expensive as compared to enteral nutritional mixtures.

### The Use of the Catheter for Treatment, Severity Assessment, Prognosis and Monitoring of Acute Pancreatitis

It is to be noted that the catheter 100 according to any of the above-described embodiments of the present invention can be used for treatment of acute pancreatitis as well as for assessment of the severity, prognosis and monitoring of the course of acute pancreatitis.

The method for treatment of acute pancreatitis implemented by using the above-described catheter 100 provides simulation of a complex of physiological conditions disturbed due to acute inflammation at the very start of pancreatitis development by topically implementing targeted therapeutic measures. It is to be further noted that the impaired duodenal motility and the dysfunction of the major duodenal papilla play a key role in the development of the pathological inflammatory circle in acute pancreatitis. In particular, the method for treatment of acute pancreatitis implemented by using the above-described catheter 100 is a treatment technique based on the following main operations:
(1) Inflating the isolating balloons 4 so as to form a duodenal region therebetween, the formed duodenal region being isolated from the upstream and downstream sections, thus providing at least the following effects:
   a. The formation of the isolated region in the duodenum prevents the ingress of gastric content into the region, which in turn prevents acidification of the duodenum. In particular, it is to be noted that an increase in acidity (decrease in pH) in the duodenum leads to an increase in cholecystokinin production, thereby increasing the synthesis of pancreatic enzymes. However, alkaline conditions inhibit the synthesis of pancreatic enzymes. Duodenal enterokinase remains inactive at high pH, but said enzyme is the basis for activation of all pancreatic enzymes.
   b. The formation of an isolated region in the duodenum further prevents the inflow of intestinal content from the small intestine. Thus, the jejunal flora does not contaminate the duodenal content, which is an important aspect since said microbiota is pathological and should not be presented there.
(2) The functional opening 7 allows a reduced (negative) pressure in the isolated region in the duodenum to be created, the created negative pressure corresponding to the peristaltic pressure of the duodenum, wherein the functional opening 7 is exposed to the isolated region formed in the duodenum by means of the inflated balloons 4 so as to allow communication between said isolated region and the functional channel 3 having the above-described functional device connected thereto.
   a. Under normal duodenal peristalsis, a reduced pressure zone is created around the major duodenal papilla, in particular, a zone having a pressure lower than the pressure of approximately 70-100 mmAq (according to other data, up to 200 mmAq) in the ductal system of the pancreas. This is sufficient for ensuring evacuation of pancreatic juice into the intestinal lumen. The simulation of the reduced pressure being similar to physiological pressure is extremely important since it ensures the outflow of bile and pancreatic juice and provides pressure reduction in the ducts and washout thereof. The works of physiologists (Physiology of the digestive system: a university textbook. Korotko GF M.: Yurayt, 2020, 235 pp; Swain SM, Romac JM, Shahid RA, Pandol SJ, Liedtke W, Vigna SR, Liddle RA. TRPV4 channel opening mediates pressure-induced pancreatitis initiated by Piezo1 activation. J Clin Invest. 2020 May 1; 130(5):2527-2541. doi: 10.1172/JCI 134111; Cosen-Binker LI, Gaisano HY. Recent insights into the cellular mechanisms of acute pancreatitis. Can J Gastroenterol. 2007 January; 21(1): 19-24. doi: 10.1155/2007/930424) prove that bile acid reflux into the pancreatic ducts and increased intraductal pressure have been shown to lead to premature activation of enzymes within the pancreas. The negative pressure normalizes outflow of bile and pancreatic juice and causes reverse humoral regulation of the secretory function of the gland.
   b. A decrease in duodenal pressure further prevents overstretching of the intestinal wall and enterocyte death, and further prevents rupture of the bonds therebetween, which could lead to the translocation of a bacterial flora from the intestinal lumen into the retroperitoneal space and to development of septic inflammation.
(3) The protrusions 6 enclosing the functional opening 7 prevent the intestinal mucosa from adhering to the functional opening 7 and, therefore, prevent obturation of the functional channel 3. Thus, the protrusions 6 allow the negative pressure to be maintained in the isolated region in the duodenum and the duodenum content to be intaken. In addition, the enclosing protrusions 6 can be further used for attaching the mesh cover 16 thereto or attaching other mesh material thereto so as cover the functional opening 7, thereby additionally preventing the functional opening 7 from being obturated by the intestinal mucosa.
(4) The functional channel 3 having the functional opening 7 exposed to the duodenal region isolated between the inflated balloons 4 allows drugs, e.g., local anti-inflammatory drugs such as hormones (e.g., budesonide), to be delivered so as to reduce the swelling of the mucosa, in particular the swelling of the major duodenal papilla, and to induce a local anti-inflammatory effect. Instead of the anti-inflammatory drugs, local effect can be induced by using antibacterial drugs or introducing newly developed drugs (topical effect of targeted agents, biotherapeutic drugs, etc.). all cases, the topical administration of drugs into the isolated region in the duodenum provides the following positive effects:
   i. increased safety of drug administration due to the fact that the drugs do not travel outside the isolated region in the duodenum, and
   ii. increased drug effectiveness due to an increased iexposure time, wherein the increased iexposure time is provided due to the fact that the drugs cannot leave the usage area (i.e., outside the isolated region in the duodenum), and
   iii. improved control of dosage and exposure time due to the fact that the introduction of drugs into the isolated region in the duodenum can be alternated with an active aspiration of said drugs from the isolated region.
(5) A biomaterial (in particular, the bile and pancreatic juice) is aspirated from the isolated region in the duodenum via the functional opening 7 and diverted through the functional channel so as to allow the biomaterial to be accumulated or collected in the functional device vessel (not shown), wherein the collected biomaterial may be further analyzed for diagnosing acute pancreatitis or other forms of pancreatitis and for monitoring the course of said diseases. In particular, the analysis of the level of inflammation markers and their dynamics in the collected biomaterial provides data on the effectiveness of the therapy, and the bacteriological study of the collected biomaterial allows the role of flora in the development of complications to be examined. is to be noted that this approach may be the basis for the development of methods for the diagnosis and prognosis of a pancreatic pathology.
(6) The balloons 4 in the catheter 100 can perform several functions. The main function of the balloons 4 is forming the isolated region of the small intestine therebetween (in particular, the duodenum as one of small intestine sections) by inflating said balloons 4 with air or liquid supplied to the supply channel 2. In addition, the balloons 4 can be inflated by using a cooled solution (e.g., water, gelatin, hydroxyethylated starch, dextran, etc.), allowing a local hypothermic effect to by established to prevent the activation of enzymes and to stop the inflammation process. The above-described design features of the catheter 100 allow the balloons 4 to be placed in a close proximity to the pancreas, thereby promoting the most effective heat transfer. In particular, when the catheter 100 is placed in the small intestine, one of the balloons 4 may be placed in the area of the pancreatic head and then inflated therein, and the other balloon 4 may be placed in the area of the tail and then inflated therein, so that the hypothermic effect can substantially act on the entire volume of the inflamed hollow organ.
(7) Both balloons 4 in the catheter 100 are interconnected via a single channel, namely via the supply channel 2, thereby ensuring a uniform distribution of the pressure exerted by the inflated balloons 4 over different parts of the intestine. This prevents the hollow organ wall from overstretching (excessive stretching) and avoids compression of capillaries in the submucosal layer of the organ wall, which is important for preventing the necrosis. In addition, the communication between the balloons 4 via the supply channel 2 provides the redistribution of pressure therein, wherein it does not create an increased resistance to a peristaltic wave passing through the intestine, preventing the development of paresis or spasms.
(8) The mesh cover 16 attached to the balloons 4 or stretched over said balloons 4 forms an elastic frame, wherein the elastic frame prevents the gastrointestinal mucosa from adhering to the functional opening 7 and, therefore, prevents the functional opening 7 from being blocked. In addition, the mesh cover 16 can be pre-impregnated with drugs or agents in order to provide an extended topical treatment.
(9) Drainage of the gastrointestinal tract sections upstream and downstream from the region isolated by means of the balloons 4 is carried out by using the inlet openings 5.2 and outlet openings 5.1, wherein said inlet openings 5.2 and outlet openings 5.1 are formed in the body of the catheter 100 outside the body portion defined by the balloons 4 and communicate with the main channel 1. Thus, drainage of the upstream and downstream parts of the gastrointestinal tract prevents the content stagnation and eliminates obstruction.
(10) Nutrient media and drugs can be introduced into the stomach and/or the top portions of the jejunum in order to stimulate intestinal motility, prevent damage to the intestinal epithelium, and to prevent the bacterial and enterotoxin translocation. Administration of enteral nutrient media is carried out via the main channel 1 bypassing the duodenum, which is very important in the early days of inflammation since food ingressing into the duodenum stimulates enzyme synthesis and, therefore, worsens the course of acute pancreatitis.
(11) Inflation of the proximal balloon 4 in the duodenal bulb allows its position to be fixed and reduces the risk of its displacement and obturation of the major duodenal papilla by one of the inflated balloons 4. The distance between the balloons 4 can be selected such that their correct placement (in particular, when the proximal balloon 4 is located in the duodenal bulb) ensures that the distal balloon 4 will always be located below the major duodenal papilla and, therefore, will not obturate it.

### Use of the Catheter for the Treatment of Small Intestine Wall Lesions

The catheter 100 according to any one of the above-described embodiments can further be used for treatment of a defect, injury, or lesion in the small intestine wall, in particular, a lesion in the duodenum, jejunum, or ileum wall, i.e. a wall of any of the small intestine sections. It is to be noted that a lesion in the small intestine wall can be caused, e.g., by a body having a high kinetic energy. It is to be further noted that a lesion in the small intestine wall can lead to the formation of several problem areas, including a necrotic zone and a zone of molecular contusion.

A method for treatment of small intestine wall lesion implemented by using the above-described catheter 100 is a method for prolonged active intraluminal management of the extent of small intestine wall lesion, wherein said method may be used at various stages of care for a patient with abdominal lesion. In particular, the method for treatment of the small intestine wall lesion implemented by using the above-described catheter 100 is a treatment technique based on the following main operations:
(1) Inflating the proximal and distal balloons 4 with an air or liquid so as to form an isolated region of the small intestine therebetween, wherein the formed region contains a small intestine portion with a single or multiple injuries or lesions is located and isolated from upstream and downstream gastrointestinal tract sections. It is to be noted that the balloons 4 can be inflated using a cooled solution (e.g., water, gelatin, hydroxyethylated starch, dextran, etc.), allowing a local hypothermic effect to be established to prevent activation of enzymes and stop the inflammation process in the small intestine. Further, both balloons 4 in the catheter 100 are connected via the supply channel 2 so as to ensure a uniform distribution of the pressure over different sections of the small intestine, thereby preventing the small intestine wall from overstretching and avoiding compression of capillaries in the submucosal layer of the small intestine wall, which is important for preventing necrosis of said small intestine wall. Furthermore, the inflation of the proximal and distal balloons 4 in the small intestine allows the position of the catheter 100 to be fixed and reduces the risk of its displacement into other sections of the gastrointestinal tract, which otherwise, e.g., could lead to obturation of the major duodenal papilla.
(2) The functional opening 7 allows a reduced (negative) pressure to be created in the isolated region in the small intestine and further allows a content of the small intestine (e.g., the bile, pancreatic juice and chyme either separately or in any combination thereof) to be continuously evacuated, wherein the functional opening 7 is exposed to the isolated region formed in the small intestine by means of the inflated balloons 4 and provides the communication between the isolated region and the functional channel 3 having the above functional device (e.g., a controlled negative pressure pump) connected thereto. Furthermore, the functional opening 7 can be used for local administration of drugs or agents (e.g., antibiotics, antiseptics, radio opaque agents, etc.) into the isolated region in the small intestine. In all cases, the possibility of topical administration of drugs into the isolated region in the small intestine provides the following positive effects: (i) increased safety of administering drugs since they do not penetrate outside the isolated region in the small intestine; (ii) increased effectiveness of drugs due to an increased exposure time allowed by preventing the drug from penetrating outside the region of drug application (i.e., outside the isolated region in the small intestine); and (iii) improved drug dosage control and exposure time control due to the fact that the introduction of the drugs into the isolated region in the small intestine can be alternated with active aspiration of said drugs from said isolated region. In addition, the functional device (e.g., a controlled negative pressure pump) allows the content of the small intestine to be actively removed, so that said content is prevented from entering the abdominal cavity, thereby preventing the development of peritonitis in the patient.
(3) Stenting and fluid communication between the gastrointestinal tract sections located upstream (proximal or upstream of the balloons 4 when the catheter 100 is installed) and downstream (distal or downstream of the balloons 4 when the catheter 100 is installed) of the region isolated using the balloons 4 is carried out by means of inlet openings 5.2 and outlet openings 5.1 provided in the body of the catheter 100 outside the body portion defined by the balloons 4 and communicating with the main channel 1, which in turn provides the possibility of passive drainage of said upstream and downstream gastrointestinal tract sections, the possibility of relieving said upstream and downstream gastrointestinal tract sections and the possibility of performing enteral feeding of the patient. In particular, drainage of the upstream and downstream gastrointestinal tract sections during patient treatment prevents stagnation of the content in said sections, thus preventing intestinal obstruction, and further prevents said content from entering the injured or damaged section of the small intestine. Furthermore, enteral feeding of the patient stimulates intestinal motility and corresponds to the “fast track” technique of patient rehabilitation.
(4) The protrusions 6 enclosing the functional opening 7 prevent the mucosa of the small intestine from adhering to the functional opening 7 and, therefore, prevent obturation of the functional channel 3. Thus, the protrusions 6 allow the negative pressure to be maintained in the isolated region in the small intestine and provide the intake of the small intestine content. In addition, said enclosing protrusions 6 can be further used for attaching the mesh cover 16 or other mesh material thereto so as to cover the functional opening 7, further preventing the functional opening 7 from being obturated by the mucosa of the small intestine wall.
(5) The mesh cover 16 attached to the balloons 4 or stretched over said balloons 4 forms an elastic frame preventing the mucosa of the small intestine wall from adhering to the functional opening 7 and, therefore, resists blocking of the functional opening 7. In addition, the mesh cover 16 can be pre-impregnated with drugs or agents to provide extended topical treatment.

It is to be noted that the proposed method for treatment of small intestine wall lesion or damage implemented by using the above-described catheter 100 is in fact a modification of the “damage control” treatment tactics commonly used by military field surgeons. A key difference of the proposed method for treatment of small intestine wall lesion or damage as compared to similar prior art methods is in that the proposed method allows the removement of the injured or damaged organ to be avoided and further allows the nature of changes occurring therein over time to be assessed. In particular, whereas a prior art method for treatment of small intestine wall lesion or damage consists to a large extent in monitoring the patient following the operation, the proposed method for treatment of small intestine wall lesion or damage, in addition to the monitoring, involves the prolonged intraluminal active management of the extent of said lesion and further involves the active treatment by removing the content of the small intestine, topical administration of drugs and enteral feeding.

### Clinical Examples of Use of the Catheter for Treatment of Bleeding into an Esophageal or Intestinal Lumen and for Treatment of an Esophageal or Intestinal Wall Fistula/Perforation

Example 1. Patient 1 (male, 36 years) diagnosed with stage IV thoracic esophageal cancer was admitted with gastrointestinal hemorrhage after tumor stenting. The source of hemorrhage was the disintegrating tumor; however, as it was located under the stent, the exact location of the source of hemorrhage could not be determined. Attempts to perform argon plasma coagulation were unsuccessful. Removal of the stent would result in excessive trauma to the tumor, followed by increased hemorrhage. The use of a Blakemore-type hemostatic balloon catheter was judged to be ineffective, on the one hand, due to the need to remove the existing stent, and on the other hand, said removal would not guarantee hemostasis, not to mention the possibility of the development of concomitant complications, e.g., esophageal wall necrosis. Following a consultation of physicians, it was decided to use the catheter 100 according to the present invention, in particular, to isolate a portion of Patient 1's esophagus by inflating the isolating balloons 4, and to subsequently introduce a hemostatic polysaccharide powder into the isolated region formed between the inflated balloons 4. The inflated balloons 4 prevented migration of both the clots and the administered drug, allowing the patient to receive fluid by gavage and enteral feeding. The exposure time of the catheter 100 was two (2) days, with the results of a clinical study of the discharge extracted from the isolated region using the catheter 100 and of the clinical blood test reliably indicated the lack of recurrent hemorrhage. The catheter 100 was subsequently withdrawn and Patient 1 was discharged from the hospital, while the previously installed stent was successfully retained.

Example 2. Patient 2 (male, 54 years) was admitted for scheduled endoscopic papillosphincterotomy and retrograde cholangiography to remove common bile duct calculus. During the operation, when the major duodenal papilla was dissected, a hemorrhage developed, and attempts to clip or perform argon plasma coagulation did not succeed, while continued attempts could lead to lesion to the pancreatic duct and the development of pancreatitis. To achieve hemostasis, it was decided to use the catheter 100 according to the present invention, in particular, to isolate a region of the duodenum by means of two inflated isolating balloons 4. A hemostatic polysaccharide powder that stimulated clot formation was introduced into the isolated are formed between the isolating balloons 4. In addition, the catheter 100 allowed for drainage of the proximal and distal small intestine outside the isolated region, which allowed for monitoring of hemostasis and nutrition of the patient the following day. The following day, the catheter 100 was removed with no evidence of hemorrhage and therefore, the patient subsequently underwent lithoextraction.

Example 3. Patient 3 (male, 82 years) was admitted with colonic bleeding. From the medical history of Patient 3, it was known that a previous month, the patient underwent a right-sided hemicolectomy with the formation of ileotransversoanastomosis in connection with cancer of the ascending colon. After preliminary preparation of the colon, colonoscopy established the source of hemorrhage as extensive ulcers of the anastomosis zone. Patient 3 underwent argon plasma coagulation of mucosal lesions and sub-mucosal injection of adrenaline solution, which gave a positive effect. However, the following day, the patient developed a recurrent hemorrhage, and the same manipulations were repeated. After one day, the hemorrhage recurred. Further, colonoscopy identified ulcers as the source of hemorrhage; however, further repetition of previous therapy was associated with a risk of perforation to the colon wall. Another approach considered was laparotomy and suturing of the anastomosis region or resection thereof, but it could potentially lead to death of Patient 3 in his elderly age, so it was finally decided to use a hemostasis system based on the catheter 100 according to the present invention. Using an endoscope, the catheter 100 was introduced so that one of the two isolating balloons 4 was located in the small intestine and the other isolating balloon 4 was located in the large intestine; then both isolating balloons 4 were inflated to provide isolation of the region of intestinal anastomosis therebetween, into which the powdered hemostatic drug was subsequently introduced. In this state, the catheter 100 was left in place for two (2) days. In order to prevent stagnation in the upstream part of the small intestine, cleansing enemas were performed through the main channel 1, while gas and intestinal content were also removed using the main channel 1. The potential recurrence of hemorrhage was monitored by examining the discharge obtained using the main channel 1. Six (6) hours prior to removal of the catheter 100, liquid mesalazine was introduced into the anastomosis region, allowing to relieve inflammation. As a result, the hemorrhage was stopped and Patient 3 was eventually discharged for outpatient treatment.

Example 4. Patient 4 was admitted with fever, chest pain, and dysphagia of the fourth (4th) degree. From the medical history of Patient 4, it was known that three (3) days earlier, the patient consumed meat-based food and choked on a large piece, resulting in failure of passage of food and fluid; patient 4 did not seek help. During the examination, a piece of chicken at the level of the abdominal esophagus and cardio-esophageal junction was discovered in Patient 4 with the formation of a pressure ulcer and mediastinitis. Patient 4 underwent mediastinal drainage surgery. As a result, after four (4) days, esophageal mucus and gastric content started actively draining through the drains. Fluoroscopy revealed an esophageal fistula opening outward. An esophageal endoscopic catheter 100 according to the present invention was installed in the esophagus, wherein inflation of the isolating balloons 4 provided isolation of the fistula region. Patient 4 underwent a routine catheter 100 change 1-2 times a week, with each catheter 100 used to evacuate the content, inject contrast fluid for monitoring, and perform enteral feeding. As a result, the esophageal wall fistula closed four (4) weeks later, i.e. the wound caused by this esophageal wall lesion was healed.

Example 5. Patient 5 underwent a planned left-sided hemicolectomy with the formation of a colon anastomosis. After seven (7) days, intestinal content started draining over the control drainage. Patient 5 underwent an ultrasound examination resulting in detection of heterogeneous liquid along the left side and in the true pelvis. Relaparotomy revealed a lesion of 3×3 cm in the anterior wall in the anastomosis region. By means of an endoscopic guide, a catheter 100 according to the present invention was installed retrogradely through the rectum into the fistula region, the inflation of the isolating balloons 4 thereof providing isolation of the lesion region in the colon wall. To improve patency of the colon, cleansing enemas were performed through the main channel 1 once a day. The functional channel 3 was used to introduce antiseptic solutions into the anastomosis region and to perform sanitation. Catheter 100 was changed once (1) a week. As a result, the fistula closed after three (3) weeks, which was confirmed both radiologically and endoscopically.

Example 6. Patient 6 underwent bariatric surgery consisting of resection of a portion of the stomach and stenting of a portion of the small intestine to reduce absorption. Four (4) days later, Patient 6 developed abdominal pain and discharge was discovered to be draining from the control drain in the region of gastroenteroanastomosis. Laparoscopy in the region of gastroenteroanastomosis revealed a lesion of 1×1 cm. By means of an endoscopic guide, a catheter 100 according to the present invention was inserted, the inflation of the isolating balloons 4 thereof allowing to isolate the region of gastroenteroanastomosis and to create a reduced (negative) pressure in said isolated region. The abdominal cavity was laparoscopically sanitized. A week later, the lesion closed.

Example 7. Patient 7 underwent pancreatoduodenal resection for pancreatic head cancer. In the postoperative period, sanioserous discharge continued from the drainage established in the region of pancreatojejunoanastomosis. On the fourth (4th) day, intestinal content and bile were discovered along with the sanioserous discharge. The biochemical analysis showed amylase levels of 26000 U/l, indicating pancreatoenteroanastomosis failure. A catheter 100 according to the present invention was inserted endoscopically, the inflation of the isolating balloons 4 thereof providing isolation of the anastomosis region in which the lesion was detected therebetween. After creating reduced (negative) pressure in the isolated region, the drained discharge sharply decreased and then ceased altogether. Due to communication between the parts of the hollow organ outside the isolated region, bile entering through the hepaticoenteroanastomosis flowed freely into the distal intestines and drained. After two (2) weeks, the catheter 100 was removed. Finally, the Patient 7 was discharged from a hospital.

### Clinical Examples of Treatment, Severity Assessment, Prognosis and Monitoring of Acute Pancreatitis

Example 1. Patient 1 (male, 36 years), was admitted urgently with acute pancreatitis of moderate severity according to the Atlanta classification; Patient 1 was diagnosed with pancreatitis for the first time. The above-described catheter 100 was used for treatment of acute pancreatitis in Patient 1. Specifically, the catheter 100 was inserted into the duodenum using an endoscope to position the proximal balloon 4 behind the pyloric sphincter of the stomach and position the distal balloon 4 in the ligament of Treitz area. Balloons 4 were inflated with 60-80 ml of air supplied through the supply channel (2) to fix the catheter 100 in the duodenum of Patient 1 and form an isolated region in the duodenum between the inflated balloons 4. The functional channel 3 opening by means of the functional opening 7 formed between the balloons 4 into the isolated region in the duodenum was connected to an aspirator, wherein the aspirator was switched to a variable mode of operation, at which the fluctuation of pressure generated in the isolated region in the duodenum was 40/70 mmHg. The main channel 1 allowing for drainage of the stomach and top portion of the jejunum using inlet openings 5.2 and outlet openings 5.1 was connected to a urine receptacle to provide passive drainage of the stomach and the top portion of the jejunum. The following day, an infusion of nutrient media was initiated through the main channel 1: first, a glucose-saline solution, and an enteral nutrient mixture the day after. The negative pressure in the isolated region in the duodenum was maintained for two (2) days, and about 300-400 ml of duodenal content (i.e., a mixture of bile and pancreatic juice) was taken from the isolated region daily into the aspirator vessel via the functional channel 3, the amylase activity wherein was 20,000 U/l. It should be noted that during treatment, positive treatment dynamics were observed, and the level of blood amylase generally decreased from 1200 U/l to 80 U/l, and the manifestations of systemic inflammatory response syndrome were resolved. Specifically, on the first day, analysis of IL-6 and IL-8 levels in the mixture of bile and pancreatic juice collected from the isolated region in the duodenum showed a five (5)-fold increase compared to the upper threshold of normal values. From a diagnostic point of view, the data obtained indicated a moderate form of pancreatitis, rather than a severe form of pancreatitis, which made it possible to avoid waiting for 48 hours. On the second day, IL-6 and IL-8 levels in the mixture of bile and pancreatic juice collected from the isolated region in the duodenum decreased to normal values, indicating a positive course of the disease and prognostically indicating a reduced risk of infection. Thus, the targeted analysis of inflammation markers in the bile and pancreatic juice mixture obtained using the catheter 100 from the isolated region in the duodenum further improved the diagnosis of the severity of acute pancreatitis and predicted the course of the disease, and further allowed for the monitoring of therapy effectiveness. On the third day, peristalsis was restored, therefore the catheter 100 was removed from the duodenum and therefore, the treatment of acute pancreatitis in Patient 1 was stopped. On the tenth day, Patient 1 was discharged for outpatient treatment.

Example 2. Patient 2 (female, 45 years), was admitted 6 hours after the onset of acute pancreatitis attack, with the severity of the disease corresponding to a severe course according to the generally accepted rating scales. The above-described catheter 100 was used for treatment of acute pancreatitis in Patient 2. The catheter 100 was inserted into the duodenum using an endoscope. Balloons 4 were inflated using 80 ml of a hydroxy-starch solution cooled to 4 °C and fed through the supply channel (2) for one and a half (1.5) hours, providing an isolated region in the duodenum around the major duodenal papilla. Five mg of budesonide solution was injected into the functional channel 3 between the balloons 4 for 30 minutes, the solution supplied through the functional opening 7 into the isolated region in the duodenum. Thereafter, the functional channel 3 through which the budesonide solution had previously been introduced was connected to an aspirator that was switched to a constant operating mode, providing pressure of 70 mmHg in the isolated region in the duodenum. At the same time, the amount of the mixture of bile and pancreatic juice discharged had increased, indicating that the topical administration of drugs improved drainage of the ductal system of the pancreas and liver. The following day, in order to monitor the correct position of the catheter 100, a radiopaque agent was introduced into the functional channel 3 and was subsequently removed from the isolated region of the duodenum using the aspirator. In the course of treatment, the main channel 1 was used to ensure passive drainage of the stomach and small intestine of Patient 2, as well as to ensure enteral feeding of Patient 2. On the fourteenth (14th) day, Patient 2 was considered healed and was discharged for outpatient treatment.

Example 3. Patient 3 (male, 53 years) was admitted with mechanical jaundice caused by choledocholithiasis. After preliminary preparation for the examination and after conducting the corresponding examination, Patient 3 was subjected to endoscopic removal of calculus from bile ducts. Patient 3 underwent standard endoscopic papillosphincterotomy and endoscopic retrograde cholangiography with lithextraction. During these interventions, conductors were repeatedly inserted into the main pancreatic duct, resulting in lesion to the main pancreatic duct. To prevent the development of post-traumatic pancreatitis or acute pancreatitis, the above-described catheter 100 was inserted into the duodenum. Balloons 4 were inflated to form an isolated region in the duodenum, into which an anti-inflammatory drug was introduced by means of the functional channel 3 opening with the functional opening 7 into the isolated region in the duodenum. The functional channel 3 through which the anti-inflammatory drug had been previously introduced was then connected to the aspirator to provide negative pressure in the isolated region in the duodenum, allowing for evacuation of bile along with pancreatic juice from the duodenum. In the first hours, Patient 3 experienced a 5-fold increase in amylase levels, with all parameters normalizing afterwards. The secretions collected from the isolated region in the duodenum were sent for bacteriological examination with negative results, thus indicating the absence of infection. Patient 3 was discharged from the hospital on the fifth (5th) day.

Example 4. Patient 4 (female, 62 years) was admitted with acute biliary pancreatitis as a result of biliary calculus wedging into the terminal portion of choledochus and of pancreatic duct obstruction. Patient 4 underwent emergency magnetic resonance cholangiopancreatography, confirming the diagnosis. Patient 4 was indicated for endoscopic retrograde litho-extraction which was performed urgently. A catheter 100 of the present invention was subsequently used for treatment of acute pancreatitis. The catheter 100 was inserted into the duodenum along a suture guide using a C-arm. Balloons 4 were inflated with a polyglucin solution cooled to 5 °C for 2 hours to form an isolated region in the duodenum around the major duodenal papilla. To relieve edema from the large duodenal papilla and to induce a local anti-inflammatory effect in the isolated region in the duodenum, an anti-inflammatory drug budesonide was administered for 30 minutes through the functional channel 3 opening with the functional opening 7 into the isolated region in the duodenum. After 30 minutes, the polyglucin solution was removed from the balloons 4, and said balloons 4 were subsequently inflated again, this time with air, through the supply channel 2. The functional channel 3 was connected to the aspirator, alternately switched between a mode of operation providing negative pressure in the isolated region in the duodenum for ten (10) minutes and a mode of operation providing local administration of a hormone preparation solution for one (1) minute. Over the course of treatment, the main channel 1 was used to provide enteral feeding of Patient 4 with a nutritional mixture. Patient 4 recovered and was discharged from the hospital on the tenth (10th) day.

### Clinical Examples of the Use of the Catheter for Treating a Small Intestine Wall Lesions

Example 1. Patient 1 was admitted with a penetrating multiple shrapnel wound to abdominal organs. During the examination, hemostasis was achieved, allowing to identify multiple multi-level injuries to the small intestine loops. Patient 1 underwent nasointestinal intubation using the catheter 100 so that when catheter 100 was introduced or installed, the upper balloon 4 was placed above the proximal lesion and the lower balloon 4 was placed below the distal lesion. Subsequently, both balloons 4 were inflated with a liquid or air to form an isolated region in the small intestine therebetween, thereby enclosing the entirety of injured loops. The entire process of introducing or installing the catheter 100 took up approximately 20-45 minutes. The abdominal cavity of Patient 1 was drained by using silicone drains and then sutured layer by layer. Outside the body of Patient 1, the catheter 100 was attached to the nose, while the functional channel 3 having a functional opening 7 exposed to the small intestine region isolated between the balloons 4 was connected to a controlled negative pressure pump, thereby allowing a reduced (negative) pressure to be created in said isolated small intestine region, so that all loops of the injured small intestine were finally actively drained, and their content (bile, pancreatic juice, chymus) was withdrawn. Patient 1 was subsequently transported to a base hospital for further treatment. After 24-48 hours, an examination of the abdominal organs was performed by means of relaparotomy, and the ultimate extent of affected loops in the small intestine was determined with subsequent resection thereof and formation of intestinal anastomoses, while the abdominal cavity was washed with antiseptic solutions (aqueous chlorhexidine solution) and sutured layer by layer. To reduce the risk of failure of the anastomosis region(s), the suture region was left isolated by the catheter 100 while performing enteral feeding by using the inlet openings 5.2 and outlet openings 5.1 provided in the body of the catheter 100 outside the body portion defined by the balloons 4 and communicating with the main channel 1, wherein the main channel 1 was connected to a collection bag formed as a flexible bag for collecting a content from passive drainage of undamaged loops of the small intestine.

Example 2. Patient 2 was injured in the ligament of Treitz region, and the therapy and suturing of the small intestine wall lesion in the ligament of Treitz region led to the development of duodenal fistula and peritonitis. The catheter 100 was introduced or installed in the small intestine of Patient 2 and balloons 4 thereof were inflated to form an isolated region therebetween, wherein the fistula area or lesion area of the small intestine wall was located, and sanitation was carried out using intraluminal vacuum therapy performed by connecting a controlled negative pressure pump to the functional channel 3 opening with a functional opening 7 into said isolated region between the balloons 3 to create a reduced (negative) pressure in said isolated region in the small intestine. As a result of said treatment using the catheter 100, aggressive media consisting of gastric, pancreatic, and bile was prevented from entering both the damaged area of the small intestine wall and the abdominal cavity, and the content of the small intestine was actively aspirated using the controlled negative pressure pump. Further, Patient 2 received enteral feeding by using inlet openings 5.2 and outlet openings 5.1 formed in the body of the catheter 100 outside the body portion defined by the balloons 4 and communicating with the main channel 1. Conservative treatment including dressings, active aspiration of small intestine content from the ligament of Treitz region, antibacterial therapy, and nutritional support was carried out for 4 weeks and resulted in a secondary closure of the small intestine wall lesion.

The multi-institution open randomized trial showed that treatment of acute pancreatitis with the catheter 100 in patients with moderate to severe forms of the diseased reduced the mortality rate of said patients by a factor of 4, reduced the incidence of infectious complications by a factor of 4, reduced the risk of surgical interventions by a factor of 4, and further reduced the duration of stay of said patients in intensive care units by 1 day, reducing the cumulative duration of hospital stay by 4 days. In addition, the average cost of hospital stay was also decreased.

It is to be noted that the treatment of acute pancreatitis by using the catheter 100 essentially relates to a surgery and is the implementation and remodeling of a number of physiological processes aimed at stopping the development of inflammation and preventing the development of complications by performing the following operations implemented by using the above-described catheter 100:1) forming an region isolated from the adjacent portions of the gastrointestinal tract in the duodenum around the major duodenal papilla while maintaining communication and drainage between said adjacent parts; 2) creating reduced physiological pressure in the isolated region and carrying out active aspiration of content from said isolated region, including pancreatic juice and bile; 3) creating a local hypothermic effect on the entire surface of the pancreas; 4) providing topical administration of drugs; and 5) sampling biological media for the purpose of diagnostics, severity assessment, prognosis and monitoring of the course of acute pancreatitis.

Thus, the proposed method for treatment of small intestine wall lesion implemented by using the above-described catheter 100 provides the following positive effects: 1) prolonged isolation of the injured region of the small intestine or the injured section of the small intestine wall, 2) preventing intestinal content from entering the abdominal cavity due to active aspiration thereof from the isolated region in the small intestine, 3) the possibility of introducing drugs into the isolated region in the small intestine, and 4) nutritional support due to enteral feeding of the patient.

In case of initial detection of high-kinetic lesion to the small intestine wall in the course of medical care, the proposed method for treatment of small intestine wall lesion implemented by using the above-described catheter 100 allows loss time on eliminating the lesions in the small intestine wall to be avoided, instead of isolating the region or section containing the lesion or damage, thus ultimately providing a reduction in the medical care period from 1.5-3 hours to 20-45 minutes. Subsequently, the patient can be transported to another hospital, and a scheduled re-examination of the abdominal organs can be carried out after 24-48 hours for the purpose of final assessment of the lesion volume and subsequent surgical treatment.

Thus, the proposed method for treatment of small intestine wall lesion implemented by using the above-described catheter 100 allows the acceleration and improvement of the process of care for the wounded, reduces the incidence of gastrointestinal tract dehiscence and fistula, reduces the duration of hospital stay and rehabilitation (as it avoids management of patients using scheduled abdominal sanitation every 2-3 days), reduces the mortality rate in patients, and further reduces the cost of treatment. Furthermore, the proposed method for treatment of small intestine wall lesion implemented by using the above-described catheter 100 allows the extent of gastrointestinal organ removal to be reduced, thus improving long-term results of the patient treatment.

1-3. (canceled)

4. A method for treatment of an esophageal or intestinal wall injury, the method comprising:
inserting a catheter (100) for isolating an interior of a mammal hollow organ into a mammal esophagus or mammal intestine, the catheter comprising:
an elongate body designed to be inserted into a lumen of the mammal hollow organ;
two balloons (4) disposed along the catheter body and configured to be inflated to isolate the interior of the mammal hollow organ therebetween; and
a functional channel (3) extending in the catheter body and comprising a functional opening (7) provided in the catheter body between the balloons (4),
wherein the functional channel (3) is designed to allow:
a negative pressure to be produced in the isolated interior to take a fluid or gaseous medium therefrom via the functional opening (7); and/or
a liquid or gaseous medium to be supplied into the isolated interior via the functional opening (7); and
inflating the balloons (4) to isolate the interior of the mammal esophagus or mammal intestine therebetween; and
producing, by means of the functional channel (3) of the catheter (100), a negative pressure in the isolated interior for taking a biological content being specific for the mammal esophagus or mammal intestine therefrom via the functional opening (7) and/or supplying, by means of the functional channel (3) of the catheter (100), a liquid or gaseous medium appropriate for treatment of the esophageal or intestinal wall injury to the isolated interior via the functional opening (7).

5. The method of claim 4, wherein the catheter (100) further comprises a second channel (1) extending in the catheter body, wherein the second channel (1) is provided at opposite ends with an inlet (5.2) and an outlet (5.1) provided both in the catheter body outside a catheter part defined by the balloons (4), and wherein the method further comprises supplying an enteral nutrition mixture via the second channel (1) or performing a drainage of sections of the mammal esophagus or mammal intestine via the second channel (1), the sections being adjacent to the isolated interior.

6. A method for treatment of an esophageal or intestinal wall injury, the method comprising:
inserting a catheter (100) for isolating an interior of a mammal hollow organ into a mammal esophagus or mammal intestine, catheter comprising:
an elongate body designed to be inserted into a lumen of the mammal hollow organ;
two balloons (4) disposed along the catheter body and configured to be inflated to isolate the interior of the mammal hollow organ therebetween; and
a functional channel (3) extending in the catheter body and comprising a functional opening (7) provided in the catheter body between the balloons (4),
wherein the functional channel (3) is designed to allow:
a negative pressure to be produced in the isolated interior to take a fluid or gaseous medium therefrom via the functional opening (7); and/or
a liquid or gaseous medium to be supplied into the isolated interior via the functional opening (7); and
wherein the catheter body is further provided with a net or enclosure (16) being permeable to a fluid and/or gaseous medium, wherein the net or enclosure (16) at least partly encloses a catheter part defined by the balloons (4) so as to cover the functional opening (7), and wherein the net or enclosure (16) is attached to the balloons (4) or encloses them such that the net or enclosure (16) becomes strained when the balloons (4) are inflated,
inflating the balloons (4) to isolate the interior of the mammal esophagus or mammal intestine therebetween; and
producing, by means of the functional channel (3) of the catheter (100), a negative pressure in the isolated interior for taking a biological content being specific for the mammal esophagus or mammal intestine therefrom via the functional opening (7) and/or supplying, by means of the functional channel (3) of the catheter (100), a liquid or gaseous medium appropriate for treatment of the esophageal or intestinal wall injury to the isolated interior via the functional opening (7).

7. The method of claim 6, wherein the catheter (100) further comprises a second channel (1) extending in the catheter body, wherein the second channel (1) is provided at opposite ends with an inlet (5.2) and an outlet (5.1) provided both in the catheter body outside the catheter part defined by the balloons (4), and wherein the method further comprises supplying an enteral nutrition mixture via the second channel (1) or performing a drainage of sections of the mammal esophagus or mammal intestine via the second channel (1), the sections being adjacent to the isolated interior.

8. A method for treatment of an esophageal or intestinal wall injury, the method comprising:
inserting a catheter (100) for isolating an interior of a mammal hollow organ into a mammal esophagus or mammal intestine, the catheter comprising:
an elongate body designed to be inserted into a lumen of the mammal hollow organ;
two balloons (4) disposed along the catheter body and configured to be inflated to isolate the interior of the mammal hollow organ therebetween; and
a functional channel (3) extending in the catheter body and comprising a functional opening (7) provided in the catheter body between the balloons (4),
wherein the functional channel (3) is designed to allow:
a negative pressure to be produced in the isolated interior to take a fluid or gaseous medium therefrom via the functional opening (7); and/or
a liquid or gaseous medium to be supplied into the isolated interior via the functional opening (7); and
wherein the functional opening (7) is positioned between two ring-shaped enclosing projections (6) provided on the catheter body between the balloons (4),
inflating the balloons (4) to isolate the interior of the mammal esophagus or mammal intestine therebetween; and
producing, by means of the functional channel (3) of the catheter (100), a negative pressure in the isolated interior for taking a biological content being specific for the mammal esophagus or mammal intestine therefrom via the functional opening (7) and/or supplying, by means of the functional channel (3) of the catheter (100), a liquid or gaseous medium appropriate for treatment of the esophageal or intestinal wall injury to the isolated interior via the functional opening (7).

9. The method of claim 8, wherein the catheter (100) further comprises a second channel (1) extending in the catheter body, wherein the second channel (1) is provided at opposite ends with an inlet (5.2) and an outlet (5.1) provided both in the catheter body outside a catheter part defined by the balloons (4), and wherein the method further comprises supplying an enteral nutrition mixture via the second channel (1) or performing a drainage of sections of the mammal esophagus or mammal intestine via the second channel (1), the sections being adjacent to the isolated interior.

10. A method for treatment of bleeding into an esophageal or intestinal lumen, the method comprising:
inserting a catheter (100) for isolating an interior of a mammal hollow organ into a mammal esophagus or mammal intestine, the catheter comprising:
an elongate body designed to be inserted into a lumen of the mammal hollow organ;
two balloons (4) disposed along the catheter body and configured to be inflated to isolate the interior of the mammal hollow organ therebetween; and
a functional channel (3) extending in the catheter body and comprising a functional opening (7) provided in the catheter body between the balloons (4),
wherein the functional channel (3) is designed to allow:
a negative pressure to be produced in the isolated interior to take a fluid or gaseous medium therefrom via the functional opening (7); and/or
a liquid or gaseous medium to be supplied into the isolated interior via the functional opening (7); and
inflating the balloons (4) to isolate the interior of the mammal esophagus or mammal intestine therebetween; and
producing, by means of the functional channel (3) of the catheter (100), a negative pressure in the isolated interior for taking a biological content being specific for the mammal esophagus or mammal intestine therefrom via the functional opening (7) and/or supplying, by means of the functional channel (3) of the catheter (100), a liquid or gaseous medium appropriate for treatment of the bleeding into the esophageal or intestinal lumen to the isolated interior via the functional opening (7).

11. The method of claim 10, wherein the catheter (100) further comprises a second channel (1) extending in the catheter body, wherein the second channel (1) is provided at opposite ends with an inlet (5.2) and an outlet (5.1) provided both in the catheter body outside a catheter part defined by the balloons (4), and wherein the method further comprises supplying an enteral nutrition mixture via the second channel (1) or performing a drainage of sections of the mammal esophagus or mammal intestine via the second channel (1), the sections being adjacent to the isolated interior.

12. A method for treatment of bleeding into an esophageal or intestinal lumen, the method comprising:
inserting a catheter (100) for isolating an interior of a mammal hollow organ into a mammal esophagus or mammal intestine, catheter comprising:
an elongate body designed to be inserted into a lumen of the mammal hollow organ;
two balloons (4) disposed along the catheter body and configured to be inflated to isolate the interior of the mammal hollow organ therebetween; and
a functional channel (3) extending in the catheter body and comprising a functional opening (7) provided in the catheter body between the balloons (4),
wherein the functional channel (3) is designed to allow:
a negative pressure to be produced in the isolated interior to take a fluid or gaseous medium therefrom via the functional opening (7); and/or
a liquid or gaseous medium to be supplied into the isolated interior via the functional opening (7); and
wherein the catheter body is further provided with a net or enclosure (16) being permeable to a fluid and/or gaseous medium, wherein the net or enclosure (16) at least partly encloses a catheter part defined by the balloons (4) so as to cover the functional opening (7), and wherein the net or enclosure (16) is attached to the balloons (4) or encloses them such that the net or enclosure (16) becomes strained when the balloons (4) are inflated,
inflating the balloons (4) to isolate the interior of the mammal esophagus or mammal intestine therebetween; and
producing, by means of the functional channel (3) of the catheter (100), a negative pressure in the isolated interior for taking a biological content being specific for the mammal esophagus or mammal intestine therefrom via the functional opening (7) and/or supplying, by means of the functional channel (3) of the catheter (100), a liquid or gaseous medium appropriate for treatment of the bleeding into the esophageal or intestinal lumen to the isolated interior via the functional opening (7).

13. The method of claim 12, wherein the catheter (100) further comprises a second channel (1) extending in the catheter body, wherein the second channel (1) is provided at opposite ends with an inlet (5.2) and an outlet (5.1) provided both in the catheter body outside a catheter part defined by the balloons (4), and wherein the method further comprises supplying an enteral nutrition mixture via the second channel (1) or performing a drainage of sections of the mammal esophagus or mammal intestine via the second channel (1), the sections being adjacent to the isolated interior.

14. A method for treatment of bleeding into an esophageal or intestinal lumen, the method comprising:
inserting a catheter (100) for isolating an interior of a mammal hollow organ into a mammal esophagus or mammal intestine, the catheter comprising:
an elongate body designed to be inserted into a lumen of the mammal hollow organ;
two balloons (4) disposed along the catheter body and configured to be inflated to isolate the interior of the mammal hollow organ therebetween; and
a functional channel (3) extending in the catheter body and comprising a functional opening (7) provided in the catheter body between the balloons (4),
wherein the functional channel (3) is designed to allow:
a negative pressure to be produced in the isolated interior to take a fluid or gaseous medium therefrom via the functional opening (7); and/or
a liquid or gaseous medium to be supplied into the isolated interior via the functional opening (7); and
wherein the functional opening (7) is positioned between two ring-shaped enclosing projections (6) provided on the catheter body between the balloons (4),
inflating the balloons (4) to isolate the interior of the mammal esophagus or mammal intestine therebetween; and
producing, by means of the functional channel (3) of the catheter (100), a negative pressure in the isolated interior for taking a biological content being specific for the mammal esophagus or mammal intestine therefrom via the functional opening (7) and/or supplying, by means of the functional channel (3) of the catheter (100), a liquid or gaseous medium appropriate for treatment of the bleeding into the esophageal or intestinal lumen to the isolated interior via the functional opening (7).

15. The method of claim 14, wherein the catheter (100) further comprises a second channel (1) extending in the catheter body, wherein the second channel (1) is provided at opposite ends with an inlet (5.2) and an outlet (5.1) provided both in the catheter body outside a catheter part defined by the balloons (4), and wherein the method further comprises supplying an enteral nutrition mixture via the second channel (1) or performing a drainage of sections of the mammal esophagus or mammal intestine via the second channel (1), the sections being adjacent to the isolated interior.

16. A method for treatment of acute pancreatitis, the method comprising:
inserting a catheter (100) for isolating an interior of a mammal hollow organ into a mammal esophagus or mammal intestine, the catheter comprising:
an elongate body designed to be inserted into a lumen of the mammal hollow organ;
two balloons (4) disposed along the catheter body and configured to be inflated to isolate the interior of the mammal hollow organ therebetween; and
a functional channel (3) extending in the catheter body and comprising a functional opening (7) provided in the catheter body between the balloons (4),
wherein the functional channel (3) is designed to allow:
a negative pressure to be produced in the isolated interior to take a fluid or gaseous medium therefrom via the functional opening (7); and/or
a liquid or gaseous medium to be supplied into the isolated interior via the functional opening (7); and
inflating the balloons (4) to isolate the interior of the mammal esophagus or mammal intestine therebetween; and
producing, by means of the functional channel (3) of the catheter (100), a negative pressure in the isolated interior for taking a biological content being specific for the mammal esophagus or mammal intestine therefrom via the functional opening (7) and/or supplying, by means of the functional channel (3) of the catheter (100), a liquid or gaseous medium appropriate for treatment of the acute pancreatitis to the isolated interior via the functional opening (7).

17. The method of claim 16, wherein the catheter (100) further comprises a second channel (1) extending in the catheter body, wherein the second channel (1) is provided at opposite ends with an inlet (5.2) and an outlet (5.1) provided both in the catheter body outside a catheter part defined by the balloons (4), and wherein the method further comprises supplying an enteral nutrition mixture via the second channel (1) or performing a drainage of sections of the mammal esophagus or mammal intestine via the second channel (1), the sections being adjacent to the isolated interior.

18. A method for treatment of acute pancreatitis, the method comprising:
inserting a catheter (100) for isolating an interior of a mammal hollow organ into a mammal esophagus or mammal intestine, catheter comprising:
an elongate body designed to be inserted into a lumen of the mammal hollow organ;
two balloons (4) disposed along the catheter body and configured to be inflated to isolate the interior of the mammal hollow organ therebetween; and
a functional channel (3) extending in the catheter body and comprising a functional opening (7) provided in the catheter body between the balloons (4),
wherein the functional channel (3) is designed to allow:
a negative pressure to be produced in the isolated interior to take a fluid or gaseous medium therefrom via the functional opening (7); and/or
a liquid or gaseous medium to be supplied into the isolated interior via the functional opening (7); and
wherein the catheter body is further provided with a net or enclosure (16) being permeable to a fluid and/or gaseous medium, wherein the net or enclosure (16) at least partly encloses a catheter part defined by the balloons (4) so as to cover the functional opening (7), and wherein the net or enclosure (16) is attached to the balloons (4) or encloses them such that the net or enclosure (16) becomes strained when the balloons (4) are inflated,
inflating the balloons (4) to isolate the interior of the mammal esophagus or mammal intestine therebetween; and
producing, by means of the functional channel (3) of the catheter (100), a negative pressure in the isolated interior for taking a biological content being specific for the mammal esophagus or mammal intestine therefrom via the functional opening (7) and/or supplying, by means of the functional channel (3) of the catheter (100), a liquid or gaseous medium appropriate for treatment of the acute pancreatitis to the isolated interior via the functional opening (7).

19. The method of claim 18, wherein the catheter (100) further comprises a second channel (1) extending in the catheter body, wherein the second channel (1) is provided at opposite ends with an inlet (5.2) and an outlet (5.1) provided both in the catheter body outside a catheter part defined by the balloons (4), and wherein the method further comprises supplying an enteral nutrition mixture via the second channel (1) or performing a drainage of sections of the mammal esophagus or mammal intestine via the second channel (1), the sections being adjacent to the isolated interior.

20. A method for treatment of acute pancreatitis, the method comprising:
inserting a catheter (100) for isolating an interior of a mammal hollow organ into a mammal esophagus or mammal intestine, the catheter comprising:
an elongate body designed to be inserted into a lumen of the mammal hollow organ;
two balloons (4) disposed along the catheter body and configured to be inflated to isolate the interior of the mammal hollow organ therebetween; and
a functional channel (3) extending in the catheter body and comprising a functional opening (7) provided in the catheter body between the balloons
wherein the functional channel (3) is designed to allow:
a negative pressure to be produced in the isolated interior to take a fluid or gaseous medium therefrom via the functional opening (7); and/or
a liquid or gaseous medium to be supplied into the isolated interior via the functional opening (7); and
wherein the functional opening (7) is positioned between two ring-shaped enclosing projections (6) provided on the catheter body between the balloons (4),
inflating the balloons (4) to isolate the interior of the mammal esophagus or mammal intestine there between; and
producing, by means of the functional channel (3) of the catheter (100), a negative pressure in the isolated interior for taking a biological content being specific for the mammal esophagus or mammal intestine therefrom via the functional opening (7) and/or supplying, by means of the functional channel (3) of the catheter (100), a liquid or gaseous medium appropriate for treatment of the acute pancreatitis to the isolated interior via the functional opening (7).

21. The method of claim 20, wherein the catheter (100) further comprises a second channel (1) extending in the catheter body, wherein the second channel (1) is provided at opposite ends with an inlet (5.2) and an outlet (5.1) provided both in the catheter body outside a catheter part defined by the balloons (4), and wherein the method further comprises supplying an enteral nutrition mixture via the second channel (1) or performing a drainage of sections of the mammal esophagus or mammal intestine via the second channel (1), the sections being adjacent to the isolated interior.
